(19) United States
(12) Patent Application Publication
(10) Pub. No.: US 2022/0281939 A1
(43) Pub. Date: Sep. 8, 2022

(54) MODIFIED TFF2 POLYPEPTIDES

(71) Applicant: TONIX PHARMA LIMITED, DUBLIN 2 (IE)

(72) Inventors: Seth Lederman, South Dartmouth, MA (US); Bruce Daugherty, Mendham, NJ (US)

(21) Appl. No.: 17/638,761

(22) PCT Filed: Aug. 27, 2020
(86) PCT No.: PCT/IB2020/000699

§ 371 Date: Feb. 25, 2022

Related U.S. Application Data
(60) Provisional application No. 63/041,097, filed on Jun. 18, 2020.
(60) Provisional application No. 62/943,803, filed on Dec. 4, 2019.
(60) Provisional application No. 62/892,520, filed on Aug. 27, 2019.

(51) Int. Cl. C07K 14/59; C07K 14/475

(52) U.S. Cl. CPC C07K 14/59; C07K 14/4753; A61P 35/00

(57) ABSTRACT

Described herein are modified TFF2 polypeptides, compositions comprising these polypeptides and their use to treat cancer and inflammation.

### CROSS-REFERENCE TO RELATED APPLICATIONS

This application is a United States National Phase Application filed under 35 U.S.C. § 371 of International Patent Application No. PCT/IB2020/000699, filed on Aug. 27, 2020, which claims priority from U.S. Provisional Application No. 62/892,520, filed Aug. 27, 2019, U.S. Provisional Application No. 62/943,803, filed Dec. 4, 2019 and U.S. Provisional Application No. 63/041,097, filed Jun. 18, 2020, the contents of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Aug. 27, 2020, is named 104545-0046-301-SL and is 37,602 bytes in size.

### FIELD OF DISCLOSURE

The present disclosure is in the field of treating subjects with cancer and/or inflammatory conditions using modified TFF2 polypeptides.

### BACKGROUND OF THE DISCLOSURE

Trefoil Family Factor-2 (TFF2) (also known as pancreatic spasmolytic polypeptide, PSP or spasmolytic peptide, SP) is a member of the trefoil factor family of peptides. Human TFF2 is a secreted protein of 106 amino acids. Mature human TFF2 is a 12 kDa protein that contains two trefoil domains that are separated by seven residues that are highly conserved in other species including pigs. The crystal structure of porcine TFF2 has been solved (De A et al, (1994) Proc NatlAcadSci USA 91(3):1084-8). The solution structure of porcine TFF2 has been studied by NMR (Carr, M D et al, (1994) Proc Natl Acad Sci USA 91(6):2206-10). There are six conserved cysteine residues in the trefoil domain that form three intramolecular disulfide bonds resulting in three loops stacked in a three-loop structure (May FEB, et al. (2000), Gut, 46:454-459). A proportion of human TFF2 in gastric fluid is glycosylated via an N-linkage, presumably on Asn (15) (May FEB et al., Gut 2000 46(4):454-9).

TFF2 is primarily expressed in Brunner's glands in the duodenum and in human gastric antrum and has been shown to have functional roles in the stomach and intestinal lumen (Jorgenson, K. H., and Jacobsen H. E., (1982) RegulPept., 3:207-219). Gastrin has been shown to regulate the TFF2 promoter via gastrin-responsive cis-acting elements and via signaling pathways (Tu, S. et al., (2007), Am JPhysiol. Gastrointest Liver Physiol., 292(6):G1726-37). TFF2 is also found in high concentration in cells adjacent to mucosal ulcerations (Wright N. A., Poulsom R., Stamp G. W. (1990) JPathol.;162:279-284).

TFF2 deficiency in knock-out (KO) mice exacerbates colitis induced by dextran sodium sulfate (DSS) (Judd LM et al, Am J. Physiol Gatrointest Liver Physiol. (2015) 308(1):G12-24). It is thought that TFF2 protects gastrointestinal mucosa from injury by stabilizing, and bolstering mucin gels, reducing inflammation and stimulating epithelial reestablishment. Cook et al. and showed that TFF2 is expressed by lymphocytes and is active on lymphocytes (Cook et al., (1999), FEBS Lett., 456(1):155-9). Dubeykovskaya et al. showed that TFF2 is a lymphocyte activating polypeptide and serves as an activating ligand for the CXCR4 receptor (also known as C-X-C chemokine receptor type 4, fusin or CD184) (Dubeykovskaya, Z. Dubeykovskaya, A., Wang, J., (2009), J Biol Chem., 284(6):3650-62). TFF2 is also expressed in spleen and circulating TFF2 is believed to have immunoregulatory roles (Dubeykovskaya Z, et al. Nat Commun. (2016), 7:1-11).

Exogenous TFF2 has poor pharmacokinetics and is rapidly eliminated from plasma. A modified TFF2 was generated by genetically fusing the C-terminus of TFF2 with the carboxyl-terminal peptide (CTP) of human chorionic gonadotropin β subunit, and further fusing a Flag tail (TFF2-CTP-Flag). Recombinant TFF2-CTP-Flag protein has been shown to suppress colon tumor growth (Dubeykovskaya, Z. A. et al., (2019), Cancer Gene Therapy, 26:48-57). Recombinant TFF2 also has been reported to be immunosuppressive against pancreatic cancer (Sung, Gi-Ho, et al., (2018), Animal Cells and Systems, 22:6, 368-381).

TFF2 is an appealing biologic treatment for cancer as it is stable in harsh pH environments like the stomach. The tumor micro environment (TME) is known to be low pH, which can reduce the binding of other cancer agents, such as monoclonal antibodies.

### SUMMARY OF THE DISCLOSURE

The present disclosure provides for compositions of modified TFF2 polypeptides that have enhanced bioactivity, and pharmacokinetic properties, such as increased stability and/or in vivo potency.

In some embodiments, the improved properties of the disclosed modified TFF2 polypeptides are achieved using chemical modifications including PEGylation or poly (D,L-lactic-co-glycolic acid) (PLGA), and/or or polysialylation (PSA) and/or fusion proteins, including fusion proteins with C-terminal peptide (CTP) of human chorionic gonadotropin β subunit, PASylation, homo-amino acid polymers (HAP), elastin-like peptides (ELPylation), XTENylated, and combinations of these modifications.

As used herein, TFF2 polypeptides modified by PEGylation, PASylation, PLGA conjugation and/or or PSA-conjugation or fusion proteins with HAP, ELPylation, XTENylated, or CTP of human chorionic gonadotropin β subunit, and combinations of these modifications are called modified TFF2 polypeptides.

The present disclosure provides for a composition of modified TFF2 polypeptides, including PEGylated TFF2, PASylated TFF2, PLGA-modified TFF2 and/or or PSA-modified TFF2 or TFF2 fusion proteins, for example, fusion proteins with CTP-peptide, fusion proteins with HAP, or ELPylated TFF2, and combinations of these modifications and the use of these modified TFF2 polypeptides to treat cancer, hyperplasia, dysplasia, inflammatory conditions, inflammation of the digestive system and/or any of the symptoms developed in COVID-19.

As defined herein, the term an “effective amount” means an amount of a modified TFF2 polypeptide which is necessary to at least partly obtain the desired response, or to delay the onset or inhibit progression or halt altogether the onset or progression of a particular condition being treated.

In some embodiments, the modified TFF2 polypeptide is homogenous and has improved pharmacokinetic properties as compared to non-modified or native human TFF2 polypeptides.

In some embodiments the modified TFF2 polypeptide has an amino acid sequence selected from SEQ ID NO: 1, SEQ ID NO: 3 or SEQ ID NO: 6.

In certain embodiments, the modified TFF2 polypeptide has a polypeptide sequence of that has at least 90% amino acid sequence identity to the amino acid sequence of SEQ ID NO: 1, SEQ ID NO: 3, or SEQ ID NO: 6.

In some embodiments, the modified TFF2 polypeptide has at least 95% amino acid sequence identity to the amino acid sequence of SEQ ID NO: 1, SEQ ID NO: 3, or SEQ ID NO: 6.

In some embodiments, the modified TFF2 polypeptides described herein are PEGylated with a low molecular weight linear PEG.

In some embodiments, the modified TFF2 polypeptides described herein are PEGylated with a high molecular weight branched PEG.

In some embodiments, the modified TFF2 polypeptide has increased half-life in blood as compared to unmodified human TFF2 polypeptide, such as SEQ ID NO:6.

In one embodiment, PEGylated TFF2 polypeptide has increased half-life in blood as compared to an un-PEGylated human TFF2 polypeptide.

In some embodiments, the modified TFF2 polypeptides described herein are PEGylated at a specific site or sites.

In some embodiments, the modified TFF2 polypeptides described herein are PEGylated at the N-terminus.

In some embodiments, the modified TFF2 polypeptides described herein are PEGylated at the N-terminus via aldehyde-PEG chemistry.

In other embodiments, the PEGylated TFF2 polypeptides described herein re PEGylated at the C-terminus.

In some embodiments, PEGylation of the TFF2 polypeptides described herein involves free solvent exposed amines via NHS-PEG chemistry.

In some embodiments, the modified TFF2 polypeptide include a fusion protein such as a C-terminal peptide (CTP) of human chorionic gonadotropin β subunit.

In some embodiments, the modified TFF2 polypeptide is a conjugate polypeptide such as a conjugate of PLGA.

In some embodiments, disclosed herein are TFF2 polypeptide fusion polypeptides selected from one or more of the group consisting of a TFF2 albumin-fusion protein, TFF2-IgG1 fusion protein, and TFF2-affinity tag fusion protein.

In some embodiments, the modified TFF2 polypeptide is a fusion protein with a poly-histidine tag. In some embodiments, the histidine tag contains an amino-acid cleavage site. In some embodiments, the histidine tag cleavage site is selected from SEQ ID NO: 20, SEQ ID NO: 21, SEQ ID NO: 22 and SEQ ID NO:23.

In some embodiments, native TFF2 polypeptide is formed by removing a poly-histidine tag from a fusion protein of TFF2.

In some embodiments, the histidine-tag is on either the N-terminus or C-terminus of the TFF2 polypeptide.

In other embodiments, after cleavage of the histidine tag, modified TFF2 polypeptides are formed by 1) purifying the TFF2 peptide; and 2) preparing a PEGylated, polysialylated, and/or conjugate with poly (D,L-lactic-co-glycolic acid) (PLGA) of the purified modified TFF2.

In another aspect of the disclosure are modified TFF2 polypeptides that have changes to their binding domains as represented by SEQ ID NOS: 26-28 and FIG. 1.

In another aspect of the disclosure are modified TFF2 polypeptides that have changes to the receptor-biding site residues as represented by SEQ ID NOS: 29-31 and FIG. 2.

In some embodiments, the modified TFF2 peptides represented by SEQ ID NOS: 26-31 are further modified by one or more of PEGylation, polysialylation, conjugation with PLGA and/or expressed as a fusion protein, comprising fusion polypeptides selected from the group consisting of a C-terminal peptide (CTP) of human chorionic gonadotropin β subunit, a PASylated fusion polypeptide, a XTENylated fusion polypeptide, a ELPylated fusion polypeptide, and a HAPylated fusion polypeptide.

In some embodiments, the modified TFF2 peptides represented by SEQ ID NOS: 29-31 are further modified by one or more of PEGylation, polysialylation, conjugation with PLGA and/or expressed as a fusion protein, comprising fusion polypeptides selected from the group consisting of a C-terminal peptide (CTP) of human chorionic gonadotropin β subunit, a PASylated fusion polypeptide, a XTENylated fusion polypeptide, a ELPylated fusion polypeptide and/or a HAPylated fusion polypeptide. In some embodiments, these modified TFF2 polypeptides have increased half-life in blood and/or improved pharmacodynamic properties as compared to unmodified human TFF2 of SEQ ID NO: 6.

In some embodiments, when the modified TFF2 peptides represented by SEQ ID NOS: 26-31 are PEGylated, the modified TFF2 binding-domain polypeptide is PEGylated with a low molecular weight linear PEG.

In some embodiments, when the modified TFF2 peptides represented by SEQ ID NOS: 26-31 are PEGylated, the modified TFF2 binding domain polypeptide is PEGylated with a high molecular weight branched PEG.

In some embodiments, when the modified TFF2 peptides represented by SEQ ID NOS: 26-31 are PEGylated, the modified TFF2 binding-domain polypeptide is PEGylated at a specific site or sites.

In some embodiments, when the modified TFF2 peptides represented by SEQ ID NOS: 26-31 are PEGylated, the modified TFF2 binding-domain polypeptide is PEGylated at its N-terminus.

In some embodiments, when the modified TFF2 peptides represented by SEQ ID NOS: 26-31 are PEGylated, the modified TFF2 binding-domain polypeptide is PEGylated using PEGylation of the N-terminus via aldehyde-PEG chemistry.

In some embodiments, when the modified TFF2 peptides represented by SEQ ID NOS: 26-31 are PEGylated, the modified TFF2 binding-domain polypeptide is PEGylated at its C-terminus.

In some embodiments, when the modified TFF2 peptides represented by SEQ ID NOS: 26-31 are PEGylated, the PEGylation involves free solvent exposed amines via NHS-PEG chemistry.

In some embodiments, the modified TFF2 peptides described herein are glycosylated.

In some embodiments, the modified TFF2 polypeptides described herein are in a homogenous composition.

In some embodiments, the modified TFF2 polypeptides described herein are in a pharmaceutical composition which may contain one or more excipients.

In some embodiments, the pharmaceutical composition is a homogeneous population of a modified TFF2 polypeptide selected from the group consisting of a modified TFF2 polypeptide that is PEGylated, polysialylated, conjugated with PLGA, or a fusion polypeptide with CTP of human chorionic gonadotropin β subunit, PASylated, XTENylated, ELPylated, HAPylated versions or combinations of these modifications.

An aspect of the disclosure are methods of treating cancer in a subject in need of treatment, the method comprising administering to the subject a therapeutically effective amount of one or more modified TFF2 polypeptides as disclosed herein, thereby treating the cancer.

In an embodiment of the disclosure, the cancer is a cancer of the digestive system, for example, without limitation, mouth cancer, pharynx cancer, oropharynx, esophageal cancer, stomach cancer, small intestine, large intestine cancer, colon cancer, rectal cancer, anal cancer, gastric cancer, liver cancer, pancreatic cancer, gall bladder cancer, or colon cancer.

In some embodiments, the cancer treated is oropharynx cancer.

In some embodiments, the cancer treated is esophageal cancer.

In some embodiments, the cancer treated is gastric cancer.

In some embodiments, the cancer treated is pancreatic cancer.

In some embodiments, the cancer treated is colon cancer.

In some embodiments, the cancer treated is rectal cancer.

In some embodiments, the cancer treated is anal cancer.

In some embodiments, the cancer treated is liver cancer.

In some embodiments, the cancer treated is a metastatic cancer.

In some embodiments, the cancer treated is also treated with a blocking antibody to PD-1 (programmed cell death protein 1, CD279), PD-L1 (programmed death-ligand 1, CD274, or B7 homolog 1 [B7-H1]), and/or CTLA-4.

In yet another embodiment, disclosure herein is a method of treating cancer in a subject in need of treatment wherein the cancer is non-responsive to treatment with a blocking antibody to PD-1, PD-L1, or CTLA-4; wherein the subject is treated with one or more of the modified TFF2 polypeptides described herein, wherein after treatment with the modified TFF2 polypeptide composition the subject's cancer becomes susceptible to treatment with a blocking antibody to PD-1, PD-L1, or CTLA-4; and wherein the subject is subsequently treated with a blocking antibody to PD-1, PD-L1, or CTLA-4 within about 1 to about 60 days after treatment with the modified TFF2 polypeptide compositions.

In some embodiments, the modified TFF2 peptides disclosed herein can be combined with the standard-of-care for the treatment of a cancer of the digestive system. In some embodiments the modified TFF2 polypeptide is given before, concurrently or subsequently to the standard-of-care treatment.

In another aspect of the disclosure are methods for treating an inflammatory condition, such as inflammation of the digestive system in a subject in need of treatment comprising administering a modified TFF2 polypeptide to the subject.

In one embodiment, the inflammation of the digestive system is inflammatory bowel disease (IBD), including, without limitation, ulcerative colitis and Crohn's disease.

In some embodiments for treating an inflammatory condition, the modified TFF2 polypeptides disclosed herein are is administered orally, intravenously, or intramuscularly.

Another aspect of the present disclosure provides a method for treating COVID-19 or any of the complications developed in a subject in need thereof, the method comprising administering to the subject one or more of the compositions of the disclosure or one or more of the modified TFF2 polypeptides of the disclosure.

In some embodiments of any of the methods of the disclosure, the modified TFF2 polypeptides can be given before, concurrently or subsequently to the standard-of-care for treating inflammatory diseases.

The modified TFF2 polypeptides are preferably administered to an individual in a “therapeutically effective amount” or a “desired amount”, this being sufficient to show benefit to the individual.

In some embodiments of the method for treating COVID-19, the method further comprises administering an agent that inhibits or reduces SARS-CoV-2 replication.

In some embodiments of the method for treating COVID-19, the method further comprises administering an antiviral agent selected from the group consisting of ribavirin, interferon (alfacon-1), chloroquine, hydroxychloroquine, EIDD-2801, EIDD-1931, GS-5734, GS-441524, ivermectin, favipiravir, indomethacin, chlorpromazine, penciclovir, nafomostat, camostat, nitazoxanide, remdesivir, famotidine and dexamethasone.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1—depicts the chimeric recombinant modified TFF2 polypeptide Domain (D) swap peptides as disclosed in the instant application.

FIG. 2—depicts the chimeric recombinant modified TFF2 polypeptide Ligand-Binding Domain (LBD) swap peptides disclosed in the instant application.

FIG. 3A: Mice (C57BL/6 WT) received azoxymethane (AOM;10 mg/kg i.p.) followed one week later with 2.5% dextran sodium sulfate (DSS) in the drinking water for 7 days. (SAC=Sacrified). FIGS. 3B-D: AOM/DSS-treated mice formed tumors at 10 weeks and developed adenocarcinoma at 17 weeks post-AOM.

FIG. 3B: Gross images. Scale bars, 5 mm. Tumors were more frequently observed in the distal colon. FIG. 3C: Macroscopically visible tumors were counted and tumor area was measured using ImageJ Fiji. FIG. 3D: Haemotoxylin and Eosin (H&E) staining. Increased intramucosal immune cell infiltrates were detected at 10 weeks post-AOM.

FIG. 4A: Immunostaining for CD45, CD11b and PD-L1 on colon tissues from AOM/DSS-treated C57BL/6 WT mice. CD11b+myeloid cells and PD-L1 expression were increased as tumors progressed. FIGS. 4B and 4C: Immunophenotyping of intratumoral myeloid cells by flow cytometry (% of CD45+). CD11b+Gr-1+MDSCs and both granolulocytic (CD11b+Ly6G+) and monocytic (CD1 lb+Ly6G-Ly6C+) MDSC subsets were markedly increased in tumors (See FIG. 4B). Macrophages (MQ; CD11b Ly6C-F4/80+) and dendritic cells (DC; CD11c+F4/80-) (See FIG. 4C).

FIGS. 5A and 5B: Immunophenotyping of tumor-infiltrating T cells by flow cytometry (% of CD45+). The proportion of T cells was decreased as tumors develop; this decrease was driven by a reduction in CD8+T cells (FIG. 5A). CD4+CD25+Foxp3+regulatory T cells (Treg) were increased in the late stage of tumors, leading to a greater decrease in CD8+T cells to Treg ratio (FIG. 5B). FIG. 5C: Dynamics of immune cell subsets during CRC development.

FIGS. 6A to 6C: Generation of R26-LSL-Pdll-EGFP mice. Gene construct of R26-LSL-Pdll-IRES-EGFP (FIG. 6A). Endogenous GFP expression by flow cytometry (FIG. 6B) and Pdll gene expression by qPCR (FIG. 6C) in splenic CD11 b- and CD1 lb+cells in R26-PD-L1 and LysM-Cre; R26-PD-L1 mice. FIG. 6D: Experimental scheme depicting induction of CRC by AOM/DSS. FIG. 6E: Gross images of colorectal tumors at 10 weeks post-AOM. Scale bars, 5 mm. FIG. 6F: The tumor numbers were counted and tumor area measured. Note that LysM-Cre; R26-PD-L1 mice treated with AOM/DSS showed markedly enhanced early colorectal tumorigenesis.

FIGS. 7A and B: TFF2 overexpression (CD2-Tff2 mice) (FIG. 7A) and treatment with adenovirus Ad-Tff2 compared to control Ad-Fc (FIG. 7B) conferred resistance to colon carcinogenesis through suppression of MDSCs. FIG. 7C: Fusion construct Tff2-2CTP-3Flag. FIGS. 7D and 7E: TFF2-CTP-Flag prolonged the circulation time in blood (FIG. 7D) but retained bioactivity (FIG. 7E). Dubeykovskaya et al. 2016 Nat Commun. (FIGS. 7A-B); 2019 Cancer Gene Ther. (FIGS. 7C-E).

FIG. 8—Panel A: R26-PD-L1 and LysM-Cre; R26-PD-L1 mice were given AOM/DSS, and treated with fusion recombinant TFF2-CTP-Flag (300 μg i.p.) and/or anti-PD-1 (RMP1-14; 200 μg i.p.) three times a week starting at the time points indicated. Panel B: The tumor numbers counted and tumor area measured. Mice with >50% reduction of tumor area compared to control animals were defined as responders. Note that LysM-Cre; R26-PD-L1 mice (5/5; 100%) showed higher response rates to combined treatment of TFF2-CTP and anti-PD-1 than control animals (⅖; 40%).

FIG. 9—Panel A: The proportion of CD3+CD8+T cells in CD45+cells and a ratio of CD8+T cells to Treg in tumors. Note that responders had more abundant tumor-infiltrating CD8+T cells and a higher ratio of CD8+T cells to Treg. Panel B: Immunophenotyping of intratumoral myeloid cells following different treatments. A marked reduction in MDSCs, in particular M-MDSC, was observed in responders. Responders also showed a lower ratio of monocyte to MQ.

FIG. 10—SDS-PAGE (non-reducing conditions) of Protein A purification of different TFF2-HSA fusion proteins. Lane 1: Marker; lane 2: TFF2-HSA [WT]; Lane 3: TFF2-HSA [D I/I]; Lane 4: TFF2-HSA [D II/I]; Lane 5: TFF2-HSA [D II/II]; Lane 6: TFF2-HSA [LBD I/I]; Lane 7: TFF2-HSA [LBD II/I]; Lane 8: TFF2-HSA [LBD II/II].

FIG. 11—Yield of the purified TFF2-HSA fusion proteins described in FIG. 10.

### DETAILED DESCRIPTION OF THE EMBODIMENTS

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

In one embodiment, the modified TFF2 polypeptide used for PEGylation, polysialylation (PSA), or conjugation with PLGA comprises, consist of, or consist essentially of the amino acid sequence of SEQ ID NO: 1, SEQ ID NO: 3 or SEQ ID NO: 6. SEQ ID NO:1 represents human TFF2 polypeptide. The displayed sequence is further processed into a mature form (SEQ ID NO: 6). SEQ ID NO: 2 represents the human nucleotide sequence encoding TFF2, where the underscored and bolded “ATG” represents the start codon. Sequence information related to TFF2 is accessible in public databases by GenBank Accession numbers NP_005414 (protein) and NM_005423 (nucleic acid).

(SEQ ID NO: 1)
MGRRDAQLLA ALLVLGLCAL AGSEKPSPCQ CSRLSPHNRT
NCGFPGITSD QCFDNGCCFD SSVTGVPWCF HPLPKQESDQ
CVMEVSDRRN CGYPGISPEE CASRKCCFSN FIFEVPWCFF
PKSVEDCHY

With the signal peptide removed, Human TFF2 peptide has the following amino acid sequence:

Native-Human TFF2 (106 AA)

(SEQ ID NO: 6)
EKPSPCQCSRLSPHNRTNCGFPGITSDQCFDNGCCF
DSSVTGVPWCFHPLPKQESDQCVMEVSDRRNCGYPG
ISPEECASRKCCFSNFIFEVPWCFFPKSVEDCHY

SEQ ID NO: 2 is the human wild type nucleotide sequence corresponding to TFF2 (nucleotides 1-717), wherein the underscored and bolded “ATG” denotes the beginning of the open reading frame:

```
(SEQ ID NO: 2)
    1 cacggtggaa gggctggggc cacggggcag
        agaagaaagg ttatctctgc ttgttggaca
  61 aacagagggg agattataaa acatacccgg
        cagtggacac catgcattct gcaagccacc
121 ctggggtgca gctgagctag ac [symbol] ggacg
        gcgagacgcc cagctcctgg cagcgctcct
181 cgtcctgggg ctatgtgccc tggcggggag
        tgagaaaccc tccccctgcc agtgctccag
241 gctgagcccc cataacagga cgaactgcgg
        cttccctgga atcaccagtg accagtgttt
301 tgacaatgga tgctgtttcg actccagtgt
        cactggggtc ccctggtgtt tccaccccct
361 cccaaagcaa gagtcggatc agtgcgtcat
        ggaggtctca gaccgaagaa actgtggcta
421 cccgggcatc agccccgagg aatgcgcctc
        tcggaagtgc tgcttctcca acttcatctt
481 tgaagtgccc tggtgcttct tcccgaagtc
        tgtggaagac tgccattact aagagaggct
541 ggttccagag gatgcatctg gctcaccggg
        tgttccgaaa ccaaagaaga aacttcgcct
601 tatcagcttc atacttcatg aaatcctggg
        ttttcttaac catcttttcc tcattttcaa
661 tggtttaaca tataatttct ttaaataaaa
        cccttaaaat ctgctaaaaa aaaaaaa
```

In the context of the different aspects of present disclosure, the term “polypeptide” refers to a single linear chain of amino acids bonded together by peptide bonds and preferably comprises at least about 21 amino acids. A polypeptide can be one chain of a protein that is composed of more than one chain or it can be the protein itself if the protein is composed of one chain. The term “polypeptide” includes glycosylated (i.e., glycoprotein) and non-glycosylated forms of such linear chain of amino acids and mixtures of glycosylated and non-glycosylated forms.

In another embodiment, the modified TFF2 polypeptide used for PEGylation, polysialylated, or conjugated with PLGA comprises, consist of, or consist essentially of the amino acid sequence of SEQ ID NO: 3, which represents mouse TFF2 polypeptide (Accession number NP_033389).

SEQ ID NO: 3 depicts the amino acid sequence of mouse TFF2 including the signal peptide:

(SEQ ID NO: 3)
MRPRGAPLLA VVLVLGLHAL VEGEKPSPCR
CSRLTPHNRK NCGFPGITSE QCFDLGCCFD
SSVAGVPWCF HPLPNQESEQ CVMEVSARKN
CGYPGISPED CASRNCCFSN LIFEVPWCFF.
PQSVEDCHY

SEQ ID NO: 4 represent the Mus musculus TFF2 nucleotide sequence accession no. NM_009363.

(SEQ ID NO: 4)
ATTCTGCAGGCTGCCCAGGTCCAGTGGAGCAGACAT
GCGACCTCGAGGTGCCCCCCTGCTGGCAGTGGTCC
TGGTTTTGGGACTGCATGCTCTGGTAGAGGGCGAG
AAACCTTCCCCCTGTCGGTGCTCCAGGCTGACACC
CCACAACAGAAAGAACTGTGGCTTCCCGGGCATCA
CCAGTGAGCAGTGCTTTGATCTTGGATGCTGCTTT
GACTCTAGCGTCGCTGGGGTCCCTTGGTGTTTCCA
CCCACTTCCAAACCAAGAATCGGAGCAGTGTGTCA
TGGAAGTGTCAGCTCGCAAGAATTGTGGGTACCCG
GGCATCAGTCCCGAGGACTGTGCCAGTCGAAACTG
CTGCTTTTCCAACCTGATCTTTGAAGTGCCCTGGT
GTTTCTTCCCACAGTCTGTGGAAGATTGTCACTAC
TGAGAGTTGCTACTGCCGAGCCACCCGTTCCCTGG
GAGCTGCAAGCCAGAAGAAAGTTTCAACCAGACTT
CATCAATCTCTGGGGTTTCTAAAACCATCTTGACC
CTTAGCAGTGGCTAGACACAGCATTTTCCAAGTAA
AGAAAAGTTG

Methods for collection, preparation, isolation and sequencing human TFF2 is described by May FEB et al. (2000), Gut, 46:454-459, which is incorporated by reference herein.

In some embodiments, the protein/polypeptide PEGylated, polysialylated, or conjugated with PLGA can comprise a variant of SEQ ID NO: 1, SEQ ID NO: 3, or SEQ ID NO: 6 having at least from about 46% to about 50% identity to SEQ ID NOS: 1, 3, or 10, or having at least from about 50.1% to about 55% identity to SEQ ID NOS: 1, 3, or 10, or having at least from about 55.1% to about 60% identity to SEQ ID NOS: 1, 3, or 10, or having from at least about 60.1% to about 65% identity to SEQ ID NOS: 1, 3, or 10, or having from about 65.1% to about 70% identity to SEQ ID NOS: 1, 3, or 10, or having at least from about 70.1% to about 75% identity to SEQ ID NOS: 1, 3, or 10, or having at least from about 75.1% to about 80% identity to SEQ ID NOS: 1, 3, or 10, or having at least from about 80.1% to about 85% identity to SEQ ID NOS: 1, 3, or 10, or having at least from about 85.1% to about 90% identity to SEQ ID NOS: 1, 3, or 10, or having at least from about 90.1% to about 95% identity to SEQ ID NOS: 1, 3, or 10, or having at least from about 95.1% to about 97% identity to SEQ ID NOS: 1, 3, or 10, or having at least from about 97.1% to about 99% identity to SEQ ID NOS: 1, 3, or 10.

In some embodiments, the modified TFF2 polypeptide is produced from a codon optimized DNA (see, Examples 1-4).

In some embodiments, the PEGylated or PASylated modified TFF2 polypeptide is a hybrid peptide, such as without limitation, modified TFF2 polypeptide with a His-tag; TFF2-C-terminal HULG1 FC-tag, TFF2-HSA, TFF2-CTP, TFF2-CTP-FLAG, TFF2-FLAG.

In some embodiments, C-terminal peptide (CTP) of human chorionic gonadotropin is used to improve the pharmacokinetic (PK) and pharmacodynamic (PD) properties of the modified TFF2 polypeptides described herein (Calo, et al., (2015), Precision Medicine, 2:e989).

In some embodiments, the PEGylated or PASylated modified TFF2 polypeptide is truncated.

In other embodiments, the PEGylated or PASylated modified TFF2 polypeptide is glycosylated.

In some embodiments, a human PEGylated or PASylated modified TFF2 polypeptide contains conservative amino acid changes as compared to wild-type. A conservative amino acid mutation or conservative amino substitution is an amino acid replacement in a polypeptide that changes an amino acid to a different amino acid with similar biochemical properties, for example, charge, hydrophobicity and size. For example, an aliphatic amino acid can be replaced by another aliphatic amino acid etc. (see Table 1). Conservative amino acid changes can also be determined using matrices based on the Dayhoff matrix, for example, see Altschul, S F, (1991), Journal of Molecular Biology 219 (3):555-65.

TABLE 1

| | | 1-letter |
|---|---|---|
| Class | Amino acids | code |
| Aliphatic | Glycine, Alanine, Valine, Leucine, | G, A, V, |
| | Isoleucine | L, I |
| Hydroxyl or | Serine, Cysteine, Selenocysteine, | S, C, U, |
| sulfur/selenium- | Threonine, Methionine | T, M |
| containing | | |
| Cyclic | Proline | P |
| Aromatic | Phenylalanine, Tyrosine, Tryptophan | F, Y, W |
| Basic | Histidine, Lysine, Arginine | H, K, R |
| Acidic and their | Aspartate, Glutamate, Asparagine, | D, E, N, |
| amides | Glutamine | Q |

Modified TFF2 Polypeptides with Swapped Domains (D's) and Ligand Binding Domains (LBD 's)

The TFF2 structure contains two relatively symmetrical domains (DI and DII) and each domain contains two putative ligand binding domains (LBDI in DI and LBDII in D2) (see, for example, Carr et al., Proc. Natl. Acad. Sci. USA (1994), 91:2206-2210). While the identities of the ligands for LBDI and LBDII are unknown, it is possible that each binds the same ligand, or that they bind different ligands. If they bind the same ligand, it is possible that the affinities for this ligand would be different. One potential ligand for either or both LBDI and LBDII of TFF2 is the CXCR4 receptor. If TFF2 binds the CXCR4 receptor at both LBDI and LBDII, then it would lead to a complex on the cell surface with effective dimerization of two CXCR4 receptors. This type of dimerization would also be expected if LBDI and LBDII bind a common, but different receptor than CXCR4. If LBDI and LBDII each bind different ligands, then it is expected to result in effective heterodimerization of such receptors, one of which may be CXCR4.

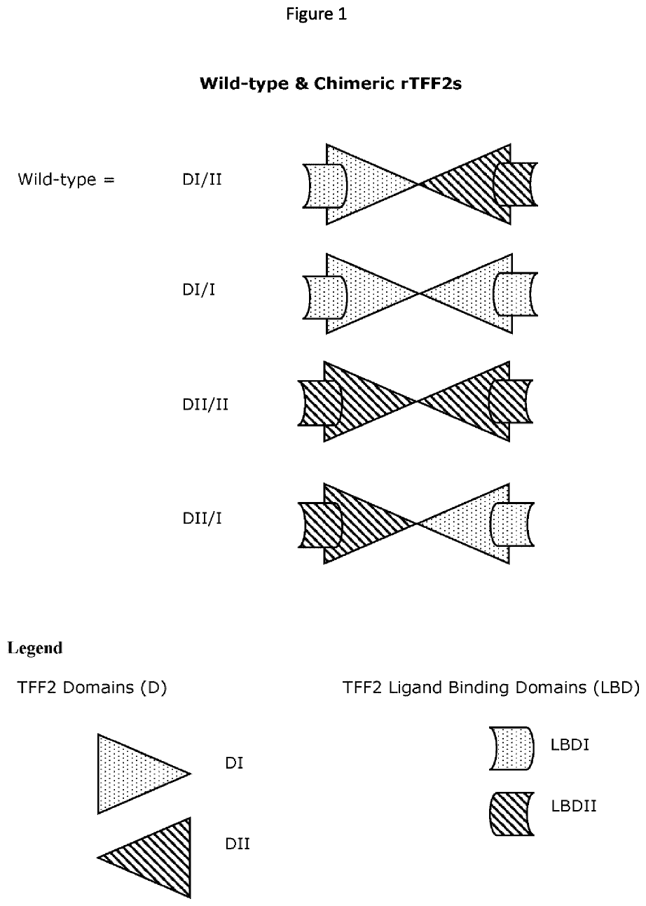

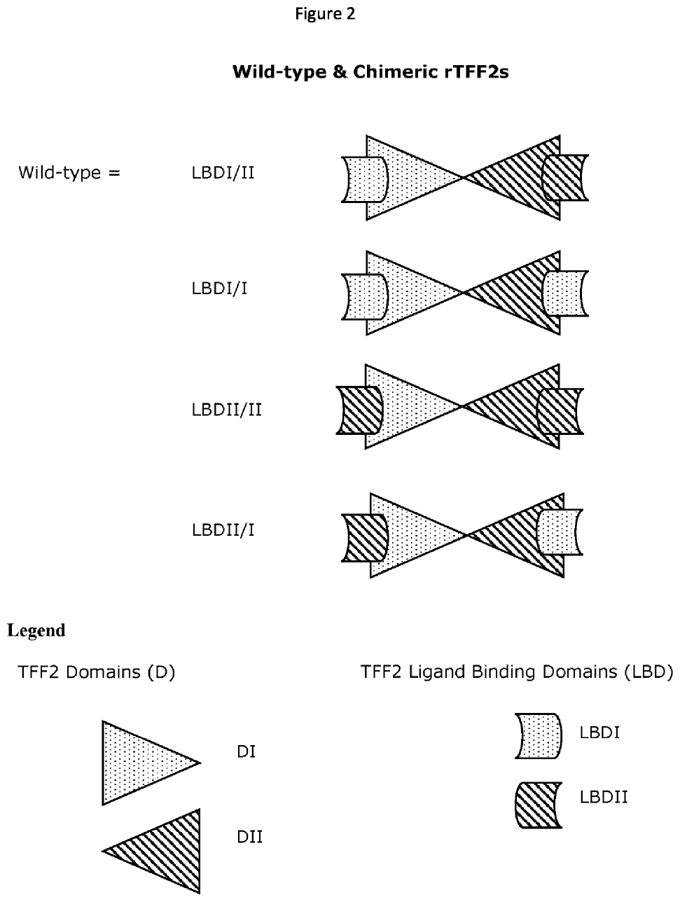

Therefore, to exploit these structural features of TFF2 and to make more potent or super-potent activators of the target ligands, including potentially CXCR4, LBD and D swapping has been employed to make new versions of TFF2 proteins, which are shown on FIGS. 1 and 2. The wild-type TFF2 is termed LBDI/II. To the extent that LBDI and LBDII interact with the same counter-receptor, but that LBDI or LBDII has greater binding avidity for the counter-receptor, then the LBD swapped domain proteins LBDI/I or LBDII/II interact with the counterreceptor with higher affinity than wild-type LBDI/II and elicit improved effects than wild-type LBDI/II. To the extent that LBDI or LBDII has a different counter-ligand, such as a receptor, than the other LBD (LBDII or LBDI, respectively), and to the extent that LBDI/II induces heterocomplexes of counter-receptors, then the LBD swapped versions (such as LBDI/I or LBDII/II, see below and FIGS. 1 and 2) induce counter-receptor homo-dimerization and elicit different and improved effects than wild-type LBDI/II. One possible counter-receptor for LBDI and LBDII that dimerizes and oligomerizes is CXCR4 (Ge B, et al., (2017) Sci Rep. 7(1):16873), such that LBDI/I or LBDII/II are more potent functional ligands of CXCR4 than wild-type TFF2 (LBDI/II). CXCR4 also forms heterodimers with the membrane bound chemokine receptors CCR5 and CCR2 (Gahbauer, S et al. (2018) PLoS Comput Biol. 14(3):e1006062). Certain modified TFF2 polypeptides, encoded by LBD swap cDNA constructs, mimic and others inhibit the function of cognate and noncognate ligands of TFF2-counter-receptors including CXCR4. Examples of ligands of CXCR4 include stromal derived factor-1 alpha (SDF-la or CXCL12), macrophage migration inhibitory factor (MIF) and extracellular ubiquitin. SDF-la is a cognate ligand of CXCR4 that binds and activates CXCR4. MIF is a non-cognate ligand of CXCR4 that triggers CXCR4 signaling (Bernhagen, J et al. (2007) Nature Medicine 13(5): 587-96). Extracellular ubiquitin is a ligand of CXCR4 (Saini, V et al. (2010) J Biol Chem 285(20) 15566; Scofield, SLC et al. (2018) Life Sci. 211:8).

In some embodiments, the modified TFF2 polypeptides contain one or more domain 1 (DI) regions of human TFF2.

In some embodiments, the modified TFF2 polypeptides contain one or more DII regions of human TFF2.

In some embodiments, the modified TFF2 polypeptides contain both DI and DII regions of human TFF2.

In some embodiments, the modified TFF2 polypeptides contain Domains with the following sequence:

Human TFF2 Domain I (residues 8-46)

(SEQ ID NO: 24)
CSRLSPHNRTNCGFPGITSDQCFDNGCCFDSSVTGVPWC

In some embodiments, the modified TFF2 polypeptides contain domains with the following sequence (see, FIG. 1).

Human TFF2 Domain II (residues 58-95)

(SEQ ID NO: 25)
CVMEVSDRRNCGYPGISPEECASRKCCFSNFIFEVPWC

In some embodiments, the modified TFF2 polypeptides contain two DI regions with the following sequence.

Human TFF2-Domain I/I Variant (D I/I, 107 AA)—two domain I regions (see FIG. 1)

(SEQ ID NO: 26)
EKPSPCQCSRLSPHNRTNCGFPGITSDQCFDNGCCFDSSVTGVPWC
FHPLPKQESDQCSRLSPHNRTNCGFPGITSDQCFDNGCCFDSSVTGV
PWCFFPKSVEDCHY

In some embodiments, modified TFF2 polypeptides contain two D II regions with the following sequence.

Human TFF2-Domain II/II Variant (D II/II, 105 AA)—two domain II regions (see FIG. 1)

(SEQ ID NO: 27)
EKPSPCQCVMEVSDRRNCGYPGISPEECASRKCCFSNFIFEVPWCFH
PLPKQESDQCVMEVSDRRNCGYPGISPEECASRKCCFSNFIFEVPWC
FFPKSVEDCHY

In some embodiments, the modified TFF2 polypeptides contain D II and DI variants, in which the order of the DI and DII are interchanged with the following sequence.

Human TFF2-Domain II/I Variant (D II/I, 106 AA)—domains I and II interchanged (see FIG. 1).

(SEQ ID NO: 28)
EKPSPCQCVMEVSDRRNCGYPGISPEECASRKCCFSNFIFEVPWCFH
PLPKQESDQCSRLSPHNRTNCGFPGITSDQCFDNGCCFDSSVTGVPW
CFFPKSVEDCHY

In some embodiments, the modified TFF2 polypeptides contain amino acid substitutions in LBD putative receptor binding site residues with the following sequence (see FIG. 2).

Human TFF2-AA-Substitutions (106 AA)—putative ligand binding domain (LBD) site residues interchanged between D I and D II (LBD II/I) (see FIG. 2).

(SEQ ID NO: 29)
EKPSPCQCSRLSPHNRTNCGYPGISSEECFDRGCCFDSSVTGVPWCF
HPLPKQESDQCVMEVSDRRNCGFPGITPDQCASNKCCFSNFIFEVPW
CFFPKSVEDCHY

In some embodiments, the modified TFF2 polypeptide contains amino acid substitutions in the receptor-binding site residues and comprises the sequence SEQ ID NO: 29. In some embodiments, the modified TFF2 polypeptide contains amino acid substitutions in the receptor-binding site residues and has the sequence SEQ ID NO: 29.

In some embodiments, the modified TFF2 polypeptides contain amino acid substitutions in LBD receptor binding site residues with the following sequence.

Human TFF2-AA-Substitutions (106 AA)—variant containing LBD putative 20 receptor binding site residues from D I only (LBD I/I) (see FIG. 2).

(SEQ ID NO: 30)
EKPSPCQCSRLSPHNRTNCGFPGITSDQCFDNGCCFDSSVTGVPWCF
HPLPKQESDQCVMEVSDRRNCGFPGITPDQCASNKCCFSNFIFEVPW
CFFPKSVEDCHY

In some embodiments, the modified TFF2 polypeptide contains amino acid substitutions in the receptor-binding site residues and comprises the sequence SEQ ID NO: 30. In some embodiments, the modified TFF2 polypeptide contains amino acid substitutions in the receptor-binding site residues and has the sequence SEQ ID NO: 30.

In some embodiments, the modified TFF2 polypeptides contain amino acid substitutions in LBD receptor binding site residues with the following sequence.

Human TFF2-AA-Substitutions (106 AA)—variant containing LBD putative receptor binding site residues from domain II only (LBD II/II) (see FIG. 2).

(SEQ ID NO: 31)
EKPSPCQCSRLSPHNRTNCGYPGISSEECFDRGCCFDSSVTGVPWCF
HPLPKQESDQCVMEVSDRRNCGYPGISPEECASRKCCFSNFIFEVPW
CFFPKSVEDCHY

In some embodiments, the modified TFF2 polypeptide contains amino acid substitutions in the receptor-binding site residues and comprises the sequence SEQ ID NO: 31. In some embodiments, the modified TFF2 polypeptide contains amino acid substitutions in the receptor-binding site residues and has the sequence SEQ ID NO: 31.

In some embodiments, modified TFF2 polypeptides with DI and DII regions have different binding affinities to counter-receptors, including CXCR4, i.e., stronger to weaker binding affinity.

In some embodiments the modified TFF2 polypeptides described herein, such as those described by SEQ ID Nos: 24-31 are modified by PEGylation, polysialylation (PSA), or conjugated with PLGA or as fusion proteins modified by PASylation, HAPylation, ELPylation, CTP of human chorionic gonadotropin β subunit, and/or or and combinations of these modifications.

In some embodiments, C-terminal peptide (CTP) of human chorionic gonadotropin is used to improve the pharmacokinetic (PK) and pharmacodynamic (PD) properties of the modified TFF2 polypeptides described herein, such as those described by SEQ ID Nos: 24-32.

In some embodiments the modified TFF2 polypeptides, such as those described by SEQ ID Nos: 24-32 are glycosylated.

The potency of the modified TFF2 polypeptides with LBD and/or D-swapped regions will be tested by calcium flux, cell migration and activation of extracellular signal-related kinases (ERKs), ERK1 and ERK2. The specificity of the effect for CXCR4 will be studied by using the CXCR4 inhibitors AMD3100 or mAb 12G5. The binding of the LBD and D-swapped proteins will be assessed by their ability to block the binding of mAb 2B11. (Dubeykovskaya, Z. Dubeykovskaya, A., Wang, J., (2009), J Biol Chem., 284(6):3650-62).

Assay for phosphorylation of ERK1 2

In some embodiments, measurement of the activity TFF2 is performed by phosphorylation of ERK1/ERK2 in Jurkat human acute T cell leukemic cells by using the AlphaLISA SureFire Ultra p-ERK ½ (Thr202/Tyr2O4) assay kit by Perkin Elmer. Jurkat cells provided by ATCC are thawed and expanded according to the instructions provided by ATCC. Cells are harvested by centrifugation and resuspended in HBSS at a 10⁷ cells/mL. Cells are seeded at 4 mL of cells/well into 384-well while opaque culture plate (PerkinElmer) and incubated at 37° C. for 1-2 hours. Wild-type and variants of recombinant TFF2 in 4 μL at a concentration of 10-30 mg/mL in HBSS containing 0.1% BSA are added to the plates to stimulate the cells and incubated at 37° C. for 5-30 minutes. Cells are lysed with 2 μL/well lysis buffer, followed by the addition of 5 mL Acceptor Mix. Plates are then sealed with Topseal-A adhesive film and incubated for 1 hr at room temperature. 5 mL Donor Mix and then added to the wells under subdued light, sealed with Topseal-A adhesive film, covered with foil and incubated for 1 hr at room temperature in the dark. Plates are read on a AlphaPlex compatible plate reader using standard AlphaPlex settings. Inhibition of TFF2 stimulation of CXCR4 is performed with AMD3100 (Sigma), a small molecule antagonist of CXCR4, or the anti-CXCR4 mAbs 12G5 and 2B11 (eBioscience) for 1-2 hours at 37° C. before the addition of recombinant TFF2.

PEGylation

Protein-based drugs in some cases are problematic as therapeutics because they may be rapidly degraded and excreted from patients, resulting in frequent dosing that may increase the immunogenic potential of the molecule and also increase the cost of therapy (Dozier, J. K., and Distefano M. D., (2015), Int, J Mol. Sci., 16:25831-25864). TFF2 protein has been shown to have poor pharmacokinetics due to it poor half-life in circulation (Dubeykovskaya, Z. A. et al., (2019), Cancer Gene Therapy, 26:48-57). Proteins chemically modified with polyethylene glycol (PEG) have shown improved pharmacological properties, including increased serum half-life, improved solubility, better physical and thermal stability, protection against enzymatic degradation, increased solubility, reduced toxicity and decreased immunogenicity.

In addition to the beneficial effects of PEGylation on pharmacokinetic parameters, PEGylation itself may enhance activity. For example, PEG-IL-10 has been shown to be more efficacious against certain cancers than unPEGylated IL-10 (see, e.g., EP 206636A2).

The disclosure contemplates the use of other polymers e.g., polypropylene glycol, or polyoxyalkylenes.

An aspect of the disclosure is PEGylated modified TFF2 polypeptides such as polypeptides of SEQ ID NO: 1 or variants thereof when compared to the full length TFF2 polypeptide. Any suitable method of PEGylation may be used. PEGylation of polypeptides is known in the art, see, for example, U.S. Pat. Nos. 6,420,339; 7,610,156; 5,766,897; 7,052,686 and 7,947,473. Also see, for example, Fee, C., and Damodaran V. B., Protein PEGylation: An overview of chemistry and process consideration, European Pharmaceutical Review, Issue 1 2010.

In an embodiment of the disclosure, a modified TFF2 polypeptide is PEGylated to increase its in vivo half-life, which may occur by prolonging its circulation in plasma by decreasing its renal clearance, and/or decrease its immunogenicity. PEGylation can also increase water solubility of hydrophobic drugs and proteins.

The overall PEGylation processes used to date for protein conjugation can be broadly classified into two types, namely a solution phase batch process and an on-column fed-batch process (Fee, Conan J.; Van Alstine, James M. (2006), Chemical Engineering Science, 61 (3): 924). This involves the mixing of reagents together in a suitable buffer solution, preferably at a temperature between 4 and 6° C., followed by the separation and purification of the desired product using a suitable technique based on its physicochemical properties, including size exclusion chromatography (SEC), ion exchange chromatography (IEX), hydrophobic interaction chromatography (HIC) and membranes or aqueous two phase systems(Veronese, edited by Francesco M. (2009). “Protein conjugates purification and characterization”. PEGylated protein drugs basic science and clinical applications (Online-Ausg. ed.). Basel: Birkhauser. pp. 113-125; and Fee, Conan J. (2003), Biotechnology and Bioengineering, 82 (2): 200-6).

The choice of the suitable functional group for the PEG derivative is based on the type of available reactive group on the molecule that will be coupled to the PEG. For proteins, typical reactive amino acids include lysine, cysteine, histidine, arginine, aspartic acid, glutamic acid, serine, threonine, tyrosine. The N-terminal amino group and the C-terminal carboxylic acid can also be used as a site-specific site by conjugation with aldehyde functional polymers (Fee, Conan J.; Damodaran, Vinod B. (2012), Biopharmaceutical Production Technology. p. 199).

In some embodiments, PEGylation occurs at one or both termini of the TFF2 polypeptide. PEGs that are activated at each terminus with the same reactive moiety are known as “homobifunctional”, whereas if the functional groups present are different, then the PEG derivative is referred as “heterobifunctional” or “heterofunctional”. The chemically active or activated derivatives of the PEG polymer are prepared to attach the PEG to the desired molecule (Pasut, G.; Veronese, F. M. (2012), Journal of Controlled Release. 161 (2): 461-472.

The techniques used to form first generation PEG derivatives are generally reacting the PEG polymer with a group that is reactive with hydroxyl groups, typically anhydrides, acid chlorides, chloroformates and carbonates. In the second generation PEGylation chemistry more efficient functional groups such as aldehyde, esters, amides etc. made available for conjugation.

Heterobifunctional PEGs are useful in linking two entities, where a hydrophilic, flexible and biocompatible spacer is needed. Preferred end groups for heterobifunctional PEGs are maleimide, vinyl sulfones, pyridyl disulfide, amine, carboxylic acids and NHS esters (see, WO2011/008495).

Third generation PEGylation agents, where the polymer has been branched, Y shaped or comb shaped are available and show reduced viscosity and lack of organ accumulation (Ryan, Sinead M; Mantovani, Giuseppe; Wang, Xuexuan; Haddleton, David M; Brayden, David J (2008), Expert Opinion on Drug Delivery, 5 (4): 371-83.

In one embodiment, the PEG is covalently linked. In another embodiment, the PEG is linked to the TFF2 polypeptide at a cysteine or lysine residue. PEGylation can be achieved using several PEG attachment moieties including, but not limited to N-hydroxylsuccinimide active ester, succinimidyl propionate, maleimide, vinyl sulfone, or thiol. A PEG polymer can be linked to a TFF2 polypeptide at either a predetermined position or can be randomly linked to the TFF2 polypeptide. PEGylation can also be mediated through a peptide linker attached to a TFF2 polypeptide. That is, the PEG moiety can be attached to a peptide linker fused to an TFF2 polypeptide, where the linker provides the site (e.g., a free cysteine or lysine) for PEG attachment.

PEGylation most frequently occurs at the alpha amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General PEGylation strategies known in the art can be applied herein. PEG may be bound to a polypeptide of the present disclosure via a terminal reactive group (a “spacer”) which mediates a bond between the free amino or carboxyl groups of one or more of the polypeptide sequences and polyethylene glycol. The PEG having the spacer which may be bound to the free amino group includes N-hydroxysuccinylimide polyethylene glycol which may be prepared by activating succinic acid ester of polyethylene glycol with N-hydroxysuccinylimide. Another activated polyethylene glycol which may be bound to a free amino group is 2,4-bis(O-methoxypolyethyleneglycol)-6-chloro-s-triazine, which may be prepared by reacting polyethylene glycol monomethyl ether with cyanuric chloride. The activated polyethylene glycol which is bound to the free carboxyl group includes polyoxyethylenediamine.

Conjugation of one or more of the polypeptide sequences of the present disclosure to PEG having a spacer may be carried out by various conventional methods. For example, the conjugation reaction can be carried out in solution at a pH of from 5 to 10, at temperature from 4° C. to room temperature, for 30 minutes to 20 hours, utilizing a molar ratio of reagent to protein of from 4:1 to 30:1. Reaction conditions may be selected to direct the reaction towards producing predominantly a desired degree of substitution. In general, low temperature, low pH (e.g., about pH 5), and short reaction time tend to decrease the number of PEGs attached, whereas high temperature, neutral to high pH (e.g., about pH 7), and longer reaction time tend to increase the number of PEGs attached. Various means known in the art may be used to terminate the reaction. In some embodiments the reaction is terminated by acidifying the reaction mixture and freezing at, e.g., −20° C. PEGylation of various polypeptides is discussed in, for example, U.S. Pat. Nos. 5,252,714; 5,643,575; 5,919,455; 5,932,462; and 5,985,263.

The present disclosure also contemplates the use of PEG mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., Amunix′ XTEN technology; Mountain View, Calif.). This obviates the need for an additional conjugation step during the manufacturing process. Moreover, established molecular biology techniques enable control of the side chain composition of the polypeptide chains, allowing optimization of immunogenicity and manufacturing properties.

In certain embodiments, a hydrophilic polymer is added to the TFF2 polypeptide. A hydrophilic polymer may be linked (directly or indirectly) to a modified TFF2 polypeptide. In a specific embodiment, a linker (e.g., a 1-5, 5-10 or 1-10 amino acid linker, such as a glycine linker) is used to link a hydrophilic polymer to a modified TFF2 polypeptide. A hydrophilic polymer may be covalently or non-covalently linked to a modified TFF2 polypeptide. A hydrophilic polymer may be a basically unstructured, hydrophilic amino acid polymer that is a functional analog of PEG, poly (methacrylate), polyvinylpyrrolidone, polyvinyl alcohol, polyacrylic acid, polyacrylamides, N-(2-Hydroxypropyl) methacrylamide (HPMA), Divinyl Ether-Maleic Anhydride (DIVEMA), polyoxazoline, polyphosphates, polyphosphazenes, and derivatives of conventional PEG (e.g., hydroxy-PEG). Hydroxy-PEG is disclosed in U.S. Pat. No. 8,129,330; and US Patent Application No. 20120114742. In certain embodiments, two, three or more hydrophilic polymers are liked to a TFF2 peptide. The hydrophilic polymer(s) may be linked to the peptide at the C-terminus, N-terminus or at both the C-terminus and N-terminus of the modified TFF2 polypeptide.

As an aspect of the disclosure, modified TFF2 polypeptide can be PEGylated using a variety of methods, including 1) PEGylation of the N-terminus via aldehyde-PEG chemistry: and 2) PEGylation of free solvent exposed amines (lysines) via NHS-PEG chemistry. PEGylation via aldehyde chemistry is described by Tureck P. L., et al., (2016), Journal of Pharmaceutical Sciences, 105:460-475. PEGylation using NHS activated PEG derivatives is based on the selectivity of NHS active esters to primary amine terminals (see Fee, C. and Damodaran V. B., (2010), European Pharmaceutical Review, Issue 1).

As used herein, the term “N-terminal modified” refers to modification of a protein or peptide at its amino (N)-terminus. For example, if the modification is PEGylation, then the PEG moiety is added/linked/conjugated at one or more amino acid residues forming the first quarter of the modified TFF2 polypeptide at the N-terminus. The amino acid residues include, but are not limited to, lysine, cysteine, serine, tyrosine, histidine, phenylalanine, or arginine.

The N-terminal modified PEG-modified TFF2 polypeptide conjugate may be obtained by reacting an N-terminal amine of modified TFF2 polypeptide with an aldehyde group of PEG in the presence of a reducing agent. The reducing agent may include NaCNBH₃ and NaBH₄.

PEGs suitable for conjugation to a polypeptide sequence are generally soluble in water at room temperature, and have the general formula R(O CH₂—CH₂)ₙ O—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure. A molecular weight of the PEG used in the present disclosure is not restricted to any particular range, and examples are set forth elsewhere herein; by way of example, certain embodiments have molecular weights between 5 kDa and 20 kDa, while other embodiments have molecular weights between 4 kDa and 10 kDa.

As used herein, the term “branched” refers to a structure of a polymeric molecule, wherein the polymeric molecule is a linear polymer serving as a backbone or main chain with branches of the same basic polymer, or another polymer, extending from the main chain. This structure can be represented by monomers polymerized into linear stretches and two or more of the linear stretches of the polymeric molecule connected at one end to one or more functional groups of a small molecule, wherein the small molecule has a molecular weight of less than 1000 Dalton. Examples of branched polymeric molecules, such as branched PEG, are presented in Roberts et al., Advanced Drug Delivery Reviews, 54:459-476 (2002). Exemplary small molecules with functional groups include N-hydroxysuccinimide, maleimide, glycerine, pentaerythritol, or hexaglycerine.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods know in the art. Exemplary reaction conditions are described throughout the specification. Cation exchange chromatography may be used to separate conjugates, and a fraction is then identified which contains the conjugate having, for example, the desired number of PEGs attached, purified free from unmodified protein sequences and from conjugates having other numbers of PEGs attached.

In another embodiment, the modified TFF2 polypeptides are PEGylated with methoxyPEG (mPEG) (see, for example, Poovi G., and Damodharan, N. (2018) European Journal of Applied Sciences, 10(1):01-14).

In another embodiment, the modified TFF2 polypeptides are PEGylated with hydroxyPEG (hPEG). Hydroxy-PEG is described in U.S. Pat. No. 8,129,330; and US Patent Application No. 20120114742.

In certain embodiments, the PEGylation of a modified TFF2 polypeptide described herein or the addition of a hydrophilic polymer to a modified TFF2 polypeptide described herein increases the half-life of the peptide in vivo by 2 to 5 times, 2 to 10 times, 2 to 20 times, 2 to 25 times, 2 to 50 times, 2 to 75 times, or 2 to 100 times compared to a non-modified TFF polypeptide, as assessed by techniques known to one of skill in the art. In some embodiments, the PEGylation of a modified TFF2 polypeptide described herein or the addition of a hydrophilic polymer to a modified TFF2 polypeptide described herein increases the half-life of the peptide in vivo by 5 to 10 times, 5 to 20 times, 5 to 25 times, 5 to 50 times, 5 to 75 times, or 5 to 100 times compared to a non-modified TFF polypeptide, as assessed by techniques known to one of skill in the art. In certain embodiments, the PEGylation of a modified TFF2 polypeptide described herein or the addition of a hydrophilic polymer to a modified TFF2 polypeptide described herein increases the half-life of the peptide in vivo by 10 to 20 times, 10 to 25 times, 10 to 50 times, 10 to 75 times, or 10 to 100 times compared to a non-modified TFF polypeptide, as assessed by techniques known to one of skill in the art. In some embodiments, the PEGylation of a modified TFF2 polypeptide described herein or the addition of a hydrophilic polymer to modified TFF2 polypeptide described herein increases the half-life of the peptide in vivo by 25 times to 50 times, 25 to 75 times, or 25 to 100 times compared to a non-modified TFF polypeptide, as assessed by techniques known to one of skill in the art. In certain embodiments, the PEGylation of a modified TFF2 polypeptide described herein or the addition of a hydrophilic polymer to a modified TFF2 polypeptide described herein increases the half-life of the peptide in vivo by 50 to 75 times or 2 to 100 times as assessed by techniques known to one of skill in the art.

Other methods of increasing the stability and/or potency of therapeutic polypeptides are known in the art and are included as embodiments of the present disclosure, for example, see, Strohl, W. R., (2015), BioDrugs, 29(4):215-239.

CTP Peptide

In some embodiments, the conjugating moiety is a CTP peptide of human chorionic gonadotropin β subunit. A CTP peptide comprises a 31 amino acid residue peptide FQSSSS*KAPPPS*LPSPS*RLPGPS*DTPLPQ (SEQ ID NO: 11) in which the S* denotes O-glycosylation sites (see, e.g., Furuhashi et al., (1995) Mol Endocrinol., 9(1):54-63.

PASylation®

In some embodiments, the modified TFF2 polypeptides described herein are PASylated (see, Aghaabdollahian, S. et al., (2019) Scientific Reports,9:2978, Payne et al. (2010) Pharm. Dev. Technol., 1-18; Pisal et al. (2010) J Pharm. Sci. 99 (6), 2557-2575; Veronese. (2001) Biomaterials 22 (5), 405-417; Veronese (2009) Milestones in drug therapy (Parnham, M. J., and Bruinvels, J., Eds.) Birkhauser, Basel; U.S. Pat. Nos. 9,221,882; 9,260,494; 9,957,323; 10,081,657; 10,174,302; and 9,574,014). Each of which is incorporated herein by reference in its entirety. PASylation is reported to increase in vivo and/or in vitro stability (U.S. Pat. No. 9,260,494). PASylation is the genetic fusion of a nucleic acid encoding a polypeptide, such as the modified TFF2 polypeptides described herein with a nucleic acid encoding a PAS polypeptide. A PAS polypeptide is a hydrophilic uncharged polypeptide consisting of Pro, Ala and Ser residues. In some embodiments, the PASylated modified TFF2 polypeptides consist of about 4, about 10, about 20, about 30, about 40, about 50, about 60, about 70, about 80, about 90, about 100, about 200, about 300, about 400, about 500, or about 600, amino acids or any ranges in between, such as 4-600, 10-500, etc.

XTENylation

In some embodiments, the modified TFF2 polypeptides described herein are XTENylated. The term “XTEN™” (Amunix Operating Inc.) and/or “XTENylation” refers to largely unstructured recombinant polypeptides comprised of the amino acids A, E, G, P, S and T. XTEN can have a length of about 864 amino acids but can also be shorter (e.g. fragments of the 864 amino acid long polypeptides according to WO2010091122 A1). The term XTENylation refers to the fusion of XTEN with a target therapeutic protein (the “payload”). XTENylation serves to increase the serum-half-life of the therapeutic protein (i.e. herein, the fusion protein of present disclosure). The term “XTEN” and/or “XTENylation” also refers to an unstructured recombinant polypeptide (URP) comprising at least 40 contiguous amino acids, wherein (a) the sum of glycine (G), aspartate (D), alanine (A), serine (S), threonine (T), glutamate (E) and proline (P) residues contained in the URP, constitutes at least 80% of the total amino acids of the unstructured recombinant polypeptide, and the remainder, when present, consists of arginine or lysine, and the remainder does not contain methionine, cysteine, asparagine, and glutamine.

ELPylation

In some embodiments, the modified TFF2 polypeptides are ELPylated. The conjugating moiety is an elastin-like polypeptide (ELP). ELPylation uses ELPs, which are repeating peptide units containing sequences commonly found in elastin. (see, Yeboah A, et al., (2016), Biotechnol Bioeng 113:1617-1627). ELPylation involves the genetic fusion of a nucleic acid encoding a polypeptide of interest with a nucleic acid encoding an elastin-like polypeptide (ELPs). An ELP comprises a VPGxG repeat motif Val Pro Gly Xaa Gly (SEQ ID NO: 12) in which x is any amino acid except proline (see, WO2018/132768).

HAP (homo-amino acid polymers)

In some embodiments, the modified TFF2 polypeptides described herein are HAPylated. HAPylation is the genetic fusion of a nucleic acid encoding a polypeptide of interest with a nucleic acid encoding a glycine-rich homoamino acid polymer (HAP). In some instances, the HAP polymer comprises a (Gly₄Ser)nrepeat motif (SEQ ID NO: 13) and sometimes are about 50, 100, 150, 200, 250, 300, or more residues in length (Schlapschy, M. et al. Protein Eng Des Sel 20, 273-284). PSA (polysialylation)

In some embodiments, the modified TFF2 polypeptides described herein can be polysialylated. Polysialic acid (PSA), also known as colominic acid (CA), is a naturally occurring polysaccharide. It is a homopolymer of N-acetylneuraminic acid with a(2→8) ketosidic linkage, or a(2→9) linkages or mixtures of both, and contains vicinal diol groups at its non-reducing end. It is negatively charged and a natural constituent of the human body. PSA can be produced in bacteria (U.S. Pat. Nos. 5,846,951; 9,018,166; 10,414,793; Zhang et al., (2014), Asian Journal of Pharmaceutical Sciences, 9(2):75-81). Methods for Polysialylating polypeptides is described in U.S. Publication No. US2012/0329127.

PLGA

Conjugation with poly(D,L-lactic-co-glycolic acid) (PLGA) In some embodiments, the modified TFF2 polypeptides described herein can be conjugated with poly (D,L-lactic-co-glycolic acid) (PLGA). PGLA is charged and a natural constituent of the human body. PLGA extends plasma half-life for example of cyclic macrolide drugs including zilucoplan (Ra Pharmaceuticals technology).

Pharmaceutical Compositions and Methods of Administration

The modified TFF2 polypeptides of the disclosure can be administered in various ways. For example, the modified TFF2 polypeptide can be administered using intravenous infusion, intramuscular administration, an implantable osmotic pump, a transdermal patch, liposomes, or other modes of administration. In one embodiment, a pump can be used (see Sefton (1987) Biomed. Eng. 14:201; Buchwald et al. (1980) Surgery 88:507; Saudek et al. (1989) N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, (1983) J. Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In another embodiment, a controlled release system can be placed in proximity of the therapeutic target thus requiring only a fraction of the systemic dose (see, e.g., Goodson, in Medical Applications of Controlled Release, vol. 2, pp. 115-138 (1984)). Other controlled release systems are discussed in the review by Langer (Science (1990) 249:1527-1533). Although proteins/peptides are poorly absorbed via oral administration, delivery systems for oral administration are known in the art, for example, Wu S. et al, (2019), Journal of Pharmaceutical Sciences, 108(6):2143-2152; and Renukunita, J. et al., 10 (2013), Int. J. Pharm., 447:75-93.

In some embodiments, a modified TFF2 polypeptide can be supplied in the form of a pharmaceutical composition, comprising an isotonic excipient prepared under sufficiently sterile conditions for human administration. Choice of the excipient and any accompanying elements of the composition comprising a PEGylated TFF2 will be adapted in accordance with the route and device used for administration. In some embodiments, a composition comprising a PEGylated TFF2 polypeptide can also comprise, or be accompanied with, one or more other ingredients that facilitate the delivery or functional mobilization of the TFF2 peptide.

These methods described herein are by no means all-inclusive, and further methods to suit the specific application is understood by the ordinary skilled artisan. Moreover, the effective amount of the compositions can be further approximated through analogy to compounds known to exert the desired effect.

One aspect of the disclosure provides a method of treating cancer in a subject in need thereof comprising administering to the subject an effective amount of any one of the compositions of the disclosure or any one of the modified TFF2 polypeptides of the disclosure.

Another aspect of the disclosure provides a method of treating Inflammatory Bowel Disease in a subject in need thereof comprising administering to the subject an effective amount of any one of the compositions of the disclosure or any one of the modified TFF2 polypeptides of the disclosure.

Another aspect of the disclosure provides a method of treating COVID-19 in a subject in need thereof comprising administering to the subject an effective amount of any one of the compositions of the disclosure or any one of the modified TFF2 polypeptides of the disclosure.

In some embodiments, the COVID-19 complications or pathologies treated by the composition or polypeptides of the disclosure include, but are not limited to, fatigue, fever, shortness of breath, muscle aches, acute respiratory distress syndrome, acute respiratory failure, acute respiratory distress syndrome (ARD), pneumonia, liver injury, cardiovascular complications, neurological and neuropsychiatric complications, kidney injuries, and the like.

In one embodiment, a modified TFF2 polypeptide can be administered in combination with an agent that inhibits or reduces SARS-CoV-2 replication. In another embodiment, a modified TFF2 polypeptide can be administered in combination with an antiviral agent selected from the group consisting of ribavirin, interferon (alfacon-1), chloroquine, hydroxychloroquine, EIDD-2801, EIDD-1931, GS-5734, GS-441524, ivermectin, favipiravir, indomethacin, chlorpromazine, penciclovir, nafomostat, camostat, nitazoxanide, remdesivir, famotidine and dexamethasone.

In some embodiments, the modified TFF2 polypeptide can be given before, concurrently or subsequently to the agent that inhibits or reduces SARS-CoV-2 replication or the antiviral agent.

According to the present disclosure, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

A modified TFF2 polypeptide can be administered to the subject one time (e.g., as a single injection or deposition). Alternatively, a modified TFF2 polypeptide can be administered once or twice daily to a subject in need thereof for a period of from about 2 to about 28 days, or from about 7 to about 10 days, or from about 7 to about 15 days. It can also be administered once or twice daily to a subject for a period of 1, 2, 3, 4, 5, 6, 7, 8, 9, 10, 11, 12 times per year, or a combination thereof. Furthermore, a modified TFF2 polypeptide can be co-administrated with another therapeutic.

In one embodiment, a modified TFF2 polypeptide can be co-administrated with a chemotherapy drug. Some non-limiting examples of conventional chemotherapy drugs include: aminoglutethimide, amsacrine, asparaginase, bcg, anastrozole, bleomycin, buserelin, bicalutamide, busulfan, capecitabine, carboplatin, camptothecin, chlorambucil, cisplatin, carmustine, cladribine, colchicine, cyclophosphamide, cytarabine, dacarbazine, cyproterone, clodronate, daunorubicin, diethylstilbestrol, docetaxel, dactinomycin, doxorubicin, dienestrol, etoposide, exemestane, filgrastim, fluorouracil, fludarabine, fludrocortisone, epirubicin, estradiol, gemcitabine, genistein, estramustine, fluoxymesterone, flutamide, goserelin, leuprolide, hydroxyurea, idarubicin, levamisole, imatinib, lomustine, ifosfamide, megestrol, melphalan, interferon, irinotecan, letrozole, leucovorin, ironotecan, mitoxantrone, nilutamide, medroxyprogesterone, mechlorethamine, mercaptopurine, mitotane, nocodazole, octreotide, methotrexate, mitomycin, paclitaxel, oxaliplatin, temozolomide, pentostatin, plicamycin, suramin, tamoxifen, porfimer, mesna, pamidronate, streptozocin, teniposide, procarbazine, titanocene dichloride, raltitrexed, rituximab, testosterone, thioguanine, vincristine, vindesine, thiotepa, topotecan, tretinoin, vinblastine, trastuzumab, and vinorelbine.

In one embodiment, a modified TFF2 polypeptide can be co-administrated with a monoclonal antibody to PD-1, PD-L1 or CTLA-4. Examples of PD-1 blocking antibodies are pembrolizumab (Keytruda®), nivolumab (Opdivo®) and cemiplimab (Libtayo®). Examples of PD-L1 blocking antibodies are atezolizumab (Tecentriq®), avelumab (Bavencio®) and durvalumab (Imfinzi®). An example of a CTLA-4 blocking antibody is ipilimumab (Yervoy®).

In one embodiment, the cancer is not responsive to the blocking anti-PD-1 or anti-PD-L1 monoclonal antibody and treatment with modified TFF2 polypeptide induces responsiveness to blocking anti-PD-1, anti-PD-L1, or anti-CTLA-4 monoclonal antibody.

In one embodiment, the chemotherapy drug is an alkylating agent, a nitrosourea, an anti-metabolite, a topoisomerase inhibitor, a mitotic inhibitor, an anthracycline, a corticosteroid hormone, a sex hormone, or a targeted anti-tumor compound.

In one embodiment, a modified TFF2 polypeptide can be co-administrated with an anti-inflammatory drug. Some non-limiting examples of anti-inflammatory drugs include: anti-inflammatory steroids (corticosteroids) (e.g. prednisone), aminosalicylates (e.g., mesalazine, Asacol HD®, Delzicol®, others), balsalazide (Colazal®) and olsalazine (Dipentum), and/or non-steroidal anti-inflammatory drugs (NSAIDs) (e.g. aspirin, ibuprofen, naproxen) and immune selective anti-inflammatory derivatives (ImSAIDs). Anti-inflammatory drugs can also include antibodies or molecules that target cytokines and chemokines including, but not limited to, anti-TNFα antibodies (e.g. infliximab (Remicade®), adalimumab (Humira®), certolizumab pegol (Cimzia®), golimumab (Simponi®), etanercept (Enbrel®)), anti-IL12 antibodies, anti-IL2 antibodies (basiliximab (Simulect®), daclizumab (Zenapax®), azathioprine (Imuran®, Azasan®), 6-mercaptopurine (6-MP, Purinethol®), cyclosporine A (Sandimmune®, Neoral®), tacrolimus (Prograf®), and anti-GM-CSF antibodies. In some embodiments, a modified TFF2 polypeptide can be co-administered with natalizumab (Tysabri®), vedolizumab (Entyvio®) and ustekinumab (Stelara®). In some embodiments the modified TFF2 polypeptide is co-administered with an inhibitor of Janus Kinase 1-3, such as the small molecule Tofacitinib. In some embodiments, the modified TFF2 polypeptide can be administered with an immune system suppressor used to treat IBD, such as azathioprine (Azasan®, Imuran®), mercaptopurine (Purinethol®, Purixan®), cyclosporine (Gengraf®, Neoral®, Sandimmune®) and methotrexate (Trexall®).

In one embodiment, a modified TFF2 polypeptide can be co-administrated with radiation therapy. Some non-limiting examples of conventional radiation therapy include: external beam radiation therapy, sealed source radiation therapy, unsealed source radiation therapy, particle therapy, and radioisotope therapy.

In one embodiment, a modified TFF2 polypeptide can be co-administrated with a cancer immunotherapy. Cancer immunotherapy comprises using the immune system of the subject to treat a cancer. For example, the immune system of a subject can be stimulated to recognize and eliminate cancer cells. Some non-limiting examples of cancer immunotherapy include: cancer vaccines, therapeutic antibodies, such as monoclonal antibody therapy (e.g., Bevacizumab, Cetuximab, and Panitumumab), cell-based immunotherapy, and adoptive cell-based immunotherapy.

A modified TFF2 polypeptide may also be used in combination with surgical or other interventional treatment regimens used for the treatment disease of the digestive system.

The compositions of this disclosure can be formulated and administered to reduce the symptoms associated with a disease of the digestive system by any means that produce contact of the active ingredient with the agent's site of action in the body of a human or non-human subject. For example, the compositions of this disclosure can be formulated and administered to reduce the symptoms associated with an inflammatory disease of the digestive system, a digestive system cancer, or a dysplasia of the digestive system, or cause a decrease in cell proliferation, or a decrease in tumor growth. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

Pharmaceutical compositions for use in accordance with the disclosure can be formulated in conventional manner using one or more physiologically acceptable carriers or excipients. The therapeutic compositions of the disclosure can be formulated for a variety of routes of administration, including systemic and topical or localized administration. Techniques and formulations generally can be found in Remington's Pharmaceutical Sciences, Meade Publishing Co., Easton, Pa. (20th ed., 2000), the entire disclosure of which is herein incorporated by reference. For systemic administration, an injection is useful, including intramuscular, intravenous, intraperitoneal, and subcutaneous. For injection, the therapeutic compositions of the disclosure can be formulated in liquid solutions, for example in physiologically compatible buffers, such as PBS, Hank's solution, or Ringer's solution. In addition, the therapeutic compositions can be formulated in solid form and redissolved or suspended immediately prior to use. Lyophilized forms are also included. Pharmaceutical compositions of the present disclosure are characterized as being at least sterile and pyrogen-free. These pharmaceutical formulations include formulations for human and veterinary use.

Any of the therapeutic applications described herein can be applied to any subject in need of such therapy, including, for example, a mammal such as a dog, a cat, a cow, a horse, a rabbit, a monkey, a pig, a sheep, a goat, or a human.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). The composition must be sterile and fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyethylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols, such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the modified TFF2 polypeptide in the required amount in an appropriate solvent with one or a combination of ingredients enumerated herein, as required, followed by filtered sterilization. Dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

A composition of the disclosure can be administered to a subject in need thereof. Subjects in need thereof can include, but are not limited to, for example, a mammal such as a dog, a cat, a cow, a horse, a rabbit, a monkey, a pig, a sheep, a goat, or a human.

A composition of the disclosure can also be formulated as a sustained and/or timed-release formulation. Such sustained and/or timed release formulations can be made by sustained release means or delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556; and 5,733,566, the disclosures of which are each incorporated herein by reference. The pharmaceutical compositions of the disclosure (e.g., that have a therapeutic effect) can be used to provide slow or sustained release of one or more of the active ingredients using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable sustained release formulations known to those of ordinary skill in the art, including those described herein, can be readily selected for use with the pharmaceutical compositions of the disclosure. Single unit dosage forms suitable for oral administration, such as, but not limited to, tablets, capsules, gel-caps, caplets, or powders, that are adapted for sustained release are encompassed by the disclosure.

In the methods described herein, a modified TFF2 polypeptide, can be administered to the subject either as RNA, in conjunction with a delivery reagent, or as a nucleic acid (e.g., a recombinant plasmid or viral vector) comprising sequences which express the gene product. Suitable delivery reagents for administration of the a modified TFF2 polypeptide, include the Mirus Transit TKO lipophilic reagent; lipofectin; lipofectamine; cellfectin; or polycations (e.g., polylysine), or liposomes.

The dosage administered can be a therapeutically effective amount of the composition sufficient to result in treatment of an inflammatory disease of the digestive system, treatment of an of a digestive system cancer, a decrease in cell proliferation, a decrease in tumor growth, or treatment of dysplasia of the digestive system, and can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

In some embodiments, the effective amount of the administered modified TFF2 polypeptide is at least about 0.01 μg/kg body weight, at least about 0.025 μg/kg body weight, at least about 0.05 μg/kg body weight, at least about 0.075 g/kg body weight, at least about 0.1 μg/kg body weight, at least about 0.25 μg/kg body weight, at least about 0.5 μg/kg body weight, at least about 0.75 μg/kg body weight, at least about 1 μg/kg body weight, at least about 5 μg/kg body weight, at least about 10 g/kg body weight, at least about 25 μg/kg body weight, at least about 50 μg/kg body weight, at least about 75 μg/kg body weight, at least about 100 μg/kg body weight, at least about 150 μg/kg body weight, at least about 200 μg/kg body weight, at least about 250 μg/kg body weight, at least about 300 μg/kg body weight, at least about 350 μg/kg body weight, at least about 400 μg/kg body weight, at least about 450 μg/kg body weight, at least about 500 μg/kg body weight, at least about 550 μg/kg body weight, at least about 600 μg/kg body weight, at least about 650 μg/kg body weight, at least about 700 μg/kg body weight, at least about 750 μg/kg body weight, at least about 800 μg/kg body weight, at least about 850 μg/kg body weight, at least about 900 μg/kg body weight, at least about 950 μg/kg body weight, at least about 1000 μg/kg body weight, at least about 1500 μg/kg body weight, at least about 2000 μg/kg body weight, at least about 2500 μg/kg body weight, at least about 3000 μg/kg body weight, at least about 3500 μg/kg body weight, at least about 4000 μg/kg body weight, at least about 4500 g/kg body weight, at least about 5000 μg/kg body weight, at least about 5500 μg/kg body weight, at least about 6000 μg/kg body weight, at least about 6500 μg/kg body weight, at least about 7000 μg/kg body weight, at least about 7500 μg/kg body weight, at least about 8000 μg/kg body weight, at least about 8500 μg/kg body weight, at least about 9000 μg/kg body weight, at least about 9500 μg/kg body weight, or at least about 10000 μg/kg body weight.

In one embodiment, a modified TFF2 polypeptide is administered at least once daily. In another embodiment, a modified TFF2 polypeptide is administered at least twice daily. In some embodiments, a modified TFF2 polypeptide is administered for at least 1 week, for at least 2 weeks, for at least 3 weeks, for at least 4 weeks, for at least 5 weeks, for at least 6 weeks, for at least 8 weeks, for at least 10 weeks, for at least 12 weeks, for at least 18 weeks, for at least 24 weeks, for at least 36 weeks, for at least 48 weeks, or for at least 60 weeks. In further embodiments, a modified TFF2 polypeptide is administered in combination with a second therapeutic agent.

Toxicity and therapeutic efficacy of therapeutic compositions of the present disclosure can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapeutic agents that exhibit large therapeutic indices are useful. Therapeutic compositions that exhibit some toxic side effects can be used.

Experimental animals can be used as models for human disease. For example, mice can be used as a mammalian model system. The physiological systems that mammals possess can be found in mice, and in humans, for example. Certain diseases can be induced in mice by manipulating their environment, genome, or a combination of both. For example, the AOM/DSS mouse model is a model for human colon cancer. In another example, the DSS mouse model is a model for human colitis. Other mouse models of carcinogenesis include the two-stage DMBA/TPA model of skin cancer, the DEN/CCL4 model of liver cancer, and the H. felis/MNU model of gastric cancer. In addition, there are numerous genetically engineered models of cancer, such as the KPC model of pancreatic cancer.

Administration of a modified TFF2 polypeptide is not restricted to a single route, but may encompass administration by multiple routes. Multiple administrations may be sequential or concurrent. Other modes of application by multiple routes will be apparent to one of skill in the art.

Recombinant Proteins and Techniques

The present disclosure utilizes conventional molecular biology, microbiology, and recombinant DNA techniques available to one of ordinary skill in the art. Such techniques are well known to the skilled worker and are explained fully in the literature. See, e.g., Maniatis, Fritsch & Sambrook, “DNA Cloning: A Practical Approach,” Volumes I and II (D. N. Glover, ed., 1985); “Oligonucleotide Synthesis” (M. J. Gait, ed., 1984); “Nucleic Acid Hybridization” (B. D. Hames& S. J. Higgins, eds., 1985); “Transcription and Translation”(B. D. Hames & S. J. Higgins, eds., 1984); “Animal Cell Culture” (R. I. Freshney, ed., 1986); “Immobilized Cells and Enzymes” (TRL Press, 1986): B. Perbal, “A Practical Guide to Molecular Cloning” (1984), and Sambrook, et al., “Molecular Cloning: a Laboratory Manual” (2001).

One skilled in the art can obtain a TFF2 protein, in several ways, including, but limited to, isolating the protein via biochemical means or expressing a nucleotide sequence encoding the protein of interest by genetic engineering methods. In some embodiments, the sequence of the polynucleotide in the host cell in which the TFF2 protein will be expressed, such as human TFF2, can be optimized for expression, while still encoding the protein of SEQ ID NOs: 1 or 3. In some embodiments, the DNA encoding TFF2 can also encode amino acids useful for protein purification such as a hybrid protein with human serum albumin (HSA), a his tag, or Fc-tag and as described herein.

A modified TFF2 polypeptide, can be a fragment of a TFF2 protein, such as, e.g. for example, the TFF2 protein fragment can encompass any portion of at least about 8 consecutive amino acids of SEQ ID NO: 1, SEQ ID NO: 3, or SEQ ID NO: 6. The fragment can comprise at least about 10 consecutive amino acids, at least about 20 consecutive amino acids, at least about 30 consecutive amino acids, at least about 40 consecutive amino acids, a least about 50 consecutive amino acids, at least about 60 consecutive amino acids, at least about 70 consecutive amino acids, at least about 80 consecutive amino acids, at least about 90 consecutive amino acids, at least about 100 consecutive amino acids, at least about 110 consecutive amino acids, or at least about 120 consecutive amino acids of SEQ ID NOS: 1, 3, or 6. Fragments include all possible amino acid lengths between about 8 and 80 about amino acids, for example, lengths between about 10 and about 80 amino acids, between about 15 and about 80 amino acids, between about 20 and about 80 amino acids, between about 35 and about 80 amino acids, between about 40 and about 80 amino acids, between about 50 and about 80 amino acids, or between about 70 and about 80 amino acids.

The modified TFF2 polypeptides can be obtained in several ways, for example, without limitation, expressing a nucleotide sequence encoding the protein of interest, or fragment thereof, by genetic engineering methods.

The nucleic acid encoding the modified TFF2 polypeptide can be expressed in an expression cassette, for example, to achieve overexpression in a cell. The nucleic acids can be RNA, cDNA, cDNA-like, or a DNA of interest in an expressible format, such as an expression cassette, which can be expressed from the natural promoter or an entirely heterologous promoter. The nucleic acid of interest can encode a protein, and may or may not include introns. Any recombinant expression system can be used, including, but not limited to, bacterial, mammalian, yeast, insect, or plant cell expression systems.

Host cells transformed with a nucleic acid sequence encoding a modified TFF2 polypeptide, can be cultured under conditions suitable for the expression and recovery of the protein from cell culture. The polypeptide produced by a transformed cell can be secreted or contained intracellularly depending on the sequence and/or the vector used. Expression vectors containing a nucleic acid sequence encoding a modified TFF2 polypeptide can be designed to contain signal sequences which direct secretion of soluble polypeptide molecules encoded a modified TFF2 polypeptide through a prokaryotic or eukaryotic cell membrane. Examples of heterologous signal peptides, without limitation are shown below in Table 2.

TABLE 2
Heterologous Signal Peptides

| Origin | Amino Acid Sequence | SEQ ID NO: |
|---|---|---|
| Human Ig kappa light | MDMRVLAQLLGLLLLCFPGARA | SEQ ID NO: |
| chain | | 14 |
| Human | MKVLWAALLVTFLAGCQA | SEQ ID NO: |
| Apolipoprotein E | | 15 |
| Bovine growth | MMAAGPRTSLLLAFALLCLPWTQVVG | SEQ ID NO: |
| hormone | | 16 |
| Drosophila 68C Glue | MKLIAVTIIACILLIGFSDLALG | SEQ ID NO: |
| | | 17 |
| Human Serum | MKWVTFISLLFLFSSAYSRGVFRR | SEQ ID NO: |
| Albumin | | 18 |
| Human alpha 1B | MSMLVVFLLLWGVTWGPVTEA | SEQ ID NO: |
| glycoprotein | | 19 |

Nucleic acid sequences comprising TFF2 that encode a polypeptide can be synthesized, in whole or in part, using chemical methods known in the art. Alternatively, TFF2 protein can be produced using chemical methods to synthesize its amino acid sequence, such as by direct peptide synthesis using solid-phase techniques. Protein synthesis can either be performed using manual techniques or by automation. Automated synthesis can be achieved, for example, using Applied Biosystems 431A Peptide Synthesizer (Perkin Elmer). Optionally, fragments of TFF2 can be separately synthesized and combined using chemical methods to produce a full-length polypeptide.

A synthetic peptide can be substantially purified via high performance liquid chromatography (HPLC). The composition of a synthetic modified TFF2 polypeptide can be confirmed by amino acid analysis or sequencing. Additionally, any portion of a TFF2 amino acid sequence can be altered during direct synthesis and/or combined using chemical methods with sequences from other proteins to produce a variant modified TFF2 polypeptide or a fusion protein.

The disclosure further encompasses methods for using a protein or modified TFF2 polypeptide encoded by a nucleic acid sequence of TFF2, such as the sequences shown in SEQ ID NOS: 2 and 3. In another embodiment, the polypeptide can be modified, such as by glycosylation and/or acetylation and/or chemical reaction or coupling, and can contain one or several non-natural or synthetic amino acids. In certain embodiments, the disclosure encompasses variants of TFF2.

Fusion Proteins

One skilled in the art understands that expression of desired protein products can be based on fusion proteins. One embodiment of a modified TFF2 polypeptide is a fusion protein. One embodiment of a fusion protein is a TFF2-albumin protein. Another embodiment is a modified TFF2-IgG1 fusion protein. These fusion proteins increase serum half-life of the modified TFF2 polypeptide relative to native or recombinant TFF2. Another type of fusion protein attaches an affinity tag that is useful in purification of recombinant protein. Fusion proteins can include new sequences at either the N-terminus or the C-terminus of the TFF2 sequence. Fusion proteins can include part of the TFF2 amino acid sequence, the whole amino acid sequence or can include new sequences that link the TFF2 sequence to a fusion domain.

A common fusion protein with an affinity tag employs a poly-histidine tag. Affinity tags are often linked to the TFF2 sequence by target protease cleavage site sequence that can be cleaved with the appropriate protease (Waugh, D S. An Overview of Enzymatic Reagents for the Removal of Affinity Tags, Protein Expr Purf 2011 Dec; 80(2): 283-293). A common target protease cleavage site sequence is the target for thrombin cleavage site with the following amino acid sequence (Leu-Val-Pro-Arg-Gly-Ser) SEQ ID NO: 20. Thrombin selectively cleaves between the Arginine and Glycine residues of the cleavage site. In other cases, the affinity tag is connected by the target sequence for enterokinase, which cleaves at the recognition site (Asp-Asp-Asp-Lys) (SEQ ID NO: 21). In another embodiment, the affinity tag is connected by the target protease cleavage site sequence for the Tobacco Etch Virus (TEV). TEV Protease is a highly specific cysteine protease that recognizes the amino-acid sequences: GIu-Asn-Leu-Tyr-Phe-Gnl-Gly (SEQ ID NO: 22), or Glu-AsLeu-Tyr-Phe-Gln-Ser (SEQ ID NO: 23) and cleaves between the Gin and Gly/Ser (the P1′ position) residues. The P1 residues can also be Ala, Met, or Cys (Kapust, R. B. et al. (2002). Biochem. and Biophysical Research Comm. 294, 949-955).

In other embodiments, after the cleavage of the affinity tag, the resulting protein includes one or more amino acid residues from the cleavage site.

In some embodiments, after cleavage of the affinity tag, the resulting protein is the native protein. As an example, TAGZyme from Qiagen® is an enzymatic system for the affinity purification of recombinant proteins using his-tags and tag removal. It combines a dipeptides (DAPase, or recombinant dipeptidyl peptidase I) for exoproteolytic cleavage from the N-terminus and also potentially two accessory amnfopeptidases (Qcyclase, or plan glutamine cyclotransferase., and pGAPase, or bacterial pyroglutamyl aninopeptidase) for the complete removal of the his-tag. All three enzyrmes in the TAGZ^{v}me display a non-cleavable his-tag for removai.

In certain embodiments, fusion proteins can be PEGylated to make pharmaceutical products, including fusion proteins with sequences that enhance half-life like albumin or IgG sequences and sequences that are used as affinity tags such as his-tags and sequences that were used as linker sequences for affinity tags or for other aspects of production.

Bacterial Expression Systems

One skilled in the art understands that expression of desired protein products in prokaryotes is most often carried out in E. coli with vectors that contain constitutive or inducible promoters. Some non-limiting examples of bacterial cells for transformation include the bacterial cell line E. coli strains DH5a or MC1061/p3 (Invitrogen Corp.®, San Diego, Calif.), which can be transformed using standard procedures practiced in the art, and colonies can then be screened for the appropriate plasmid expression. In bacterial systems, a number of expression vectors can be selected. Non-limiting examples of such vectors include multifunctional E. coli cloning and expression vectors such as BLUESCRIPT (Stratagene®). Some E. coli expression vectors (also known in the art as fusion-vectors) are designed to add a number of amino acid residues, usually to the N-terminus of the expressed recombinant protein. Such fusion vectors can serve three functions: 1) to increase the solubility of the desired recombinant protein; 2) to increase expression of the recombinant protein of interest; and 3) to aid in recombinant protein purification by acting as a ligand in affinity purification. In some instances, vectors, which direct the expression of high levels of fusion protein products that are readily purified, may also be used. Some non-limiting examples of fusion expression vectors include pGEX, which fuse glutathione S-tranferase (GST) to desired protein; pcDNA 3.1/V5-His A B & C (Invitrogen Corp.®, Carlsbad, Calif) which fuse 6x-His (SEQ ID NO: 8) to the recombinant proteins of interest; pMAL (New England Biolabs®, MA) which fuse maltose E binding protein to the target recombinant protein; the E. coli expression vector pUR278 (Ruther et al., (1983) EMBO 12:1791), wherein the coding sequence may be ligated individually into the vector in frame with the lac Z coding region in order to generate a fusion protein; and pIN vectors (Inouye et al., (1985) Nucleic Acids Res. 13:3101-3109; Van Heeke et al., (1989) J. Biol. Chem. 24:5503-5509. Fusion proteins generated by the likes of the above-mentioned vectors are generally soluble and can be purified easily from lysed cells via adsorption and binding of the fusion protein to an affinity matrix. For example, fusion proteins can be purified from lysed cells via adsorption and binding to a matrix of glutathione agarose beads subsequently followed by elution in the presence of free glutathione. For example, the pGEX vectors are designed to include thrombin or factor Xa protease cleavage sites so that the cloned target can be released from the GST moiety.

Plant, Insect, and Yeast Expression Systems

Other suitable cell lines, in addition to microorganisms such as bacteria (e.g., E. coli and B. subtilis) transformed with recombinant bacteriophage DNA, plasmid DNA or cosmid DNA expression vectors containing coding sequences for a TFF2 peptide may alternatively be used to produce the molecule of interest. A non-limiting example includes plant cell systems infected with recombinant virus expression vectors (for example, tobacco mosaic virus, TMV; cauliflower mosaic virus, CaMV) or transformed with recombinant plasmid expression vectors (e.g., Ti plasmid) containing coding sequences for a modified TFF2 polypeptide. If plant expression vectors are used, the expression of sequences encoding a modified TFF2 polypeptide can be driven by any of a number of promoters. For example, viral promoters such as the 35S and 19S promoters of CaMV can be used alone or in combination with the omega leader sequence from tobacco mosaic virus TMV. Alternatively, plant promoters such as the small subunit of RUBISCO or heat shock promoters, can be used. These constructs can be introduced into plant cells by direct DNA transformation or by pathogen-mediated transfection.

An insect system also can be used to express a modified TFF2 polypeptide or fusion protein. A number of methods for expressing recombinant protein using an insect system are known in the art, for example, see Bleckmann, M. et al., (2016), Biotechnol Bioeng. 113(9): 1975-1983; Zitzmann, J. et al., Process Optimization for Recombinant Protein Expression in Insect Cells, New Insights into Cell Culture Technology; InTech; 2017; U.S. Pat. Nos. 5,194,376; 5,843,733; For example, in one such system Autographa californica nuclear polyhedrosis virus (AcNPV) is used as a vector to express foreign genes in Spodoptera frugiperda cells or in Trichoplusia virescens in Trichoplusia larvae. Sequences encoding a modified TFF2 polypeptide can be cloned into a non-essential region of the virus, such as the polyhedrin gene, and placed under control of the polyhedrin promoter. Successful insertion of the nucleic acid sequences of a modified TFF2 polypeptide will render the polyhedrin gene inactive and produce recombinant virus lacking coat protein. The recombinant viruses are then used to infect, for example, Spodoptera frugiperda (S. frugiperda) cells or Trichoplusia ni (in Trichoplusia) larvae in the grass frugiperda (S. frugiperda) cells or Trichoplusia night moth (Trichoplusia) larvae, the polypeptide of interest has been expressed by (see Engelhard, E K et al. (1994) in 10 Proc.Natl.Acad.Sci. 3224).

In another embodiment, a yeast, for example chizosaccharomyces pombe (Schizosaccharomyces pombe); Kluyveromyces (Kluyveromyces) hosts e.g., lactic acid g Lurvy yeast (K Iactis), Kluyveromyces fragilis (K.fragilis) (ATCC 12424), K. bulgaricus (K.bulgaricus) (ATCC 16045), Clostridium Kluyveromyces (K.wickerhamii) (ATCC 24178), K.waltii (ATCC 56500), Drosophila Kluyveromyces (K.drosophilarum) (ATCC 36906), K. thermotolerans (K.thermotolerans), and Kluyveromyces marxianus (K. marxianus); Yarrowia (yarrowia) (EP 402226); Pichia yeast (Pichia pastoris) (EP 183070); Candida (Candida); Trichoderma reesei (Trichodermareesei) (EP 244234); The crude Tangmaiping hold bacteria (Neurospora crassa); Schwanniomyces (Schwanniomyces) e.g. Schwanniomyces occidentalis; and filamentous fungi such as, Neurospora strain (Neurospora), Penicillium (Penicillium), cyclosporine (Tolypocladium,), and Aspergillus (Aspergillus) host, such as Aspergillus nidulans (the A. nidulans) and Niger (A.niger). Yeasts can be transformed with recombinant yeast expression vectors containing coding sequences for a modified TFF2 polypeptide. A preferred embodiment is expression in yeast, including S cerevisiae, because yeast possesses the ability to glycosylate recombinant proteins and a significant proportion of human TFF2 in gastric fluid is glycosylated via an N-linkage, presumably on Asn(15), which may have functional importance for intravascular TFF2 and may increase plasma half-life (May FE et al., Gut 2000 46(4):454-9). When recombinant human TFF2 is expressed in S cerevisiae, a significant proportion of the recombinant protein is glycosylated via an N-linkage on Asn(15) (Thim L et al. FEBS Lett 1993: 318:345-52). Mammalian Expression Systems

Mammalian cells (such as BHK cells, VERO cells, CHO cells, HEK293 cells and the like) can also contain an expression vector (for example, one that harbors a nucleotide sequence encoding a modified TFF2 polypeptide) for expression of a desired product. Expression vectors containing such a nucleic acid sequence linked to at least one regulatory sequence in a manner that allows expression of the nucleotide sequence in a host cell can be introduced via methods known in the art. A number of viral-based expression systems can be used to express a modified TFF2 polypeptide in mammalian host cells. The vector can be a recombinant DNA or RNA vector, and includes DNA plasmids or viral vectors. For example, if an adenovirus is used as an expression vector, sequences encoding a modified TFF2 polypeptide can be ligated into an adenovirus transcription/translation complex comprising the late promoter and tripartite leader sequence. Insertion into a non-essential E1 or E3 region of the viral genome can be used to obtain a viable virus which is capable of expressing a modified TFF2 polypeptide in infected host cells. Transcription enhancers, such as the Rous sarcoma virus (RSV) enhancer, can also be used to increase expression in mammalian host cells. In addition, viral vectors can be constructed based on, but not limited to, adeno-associated virus, retrovirus, adenovirus, lentivirus or alphavirus.

Regulatory sequences are well known in the art, and can be selected to direct the expression of a protein or polypeptide of interest (such as a modified TFF2 polypeptide) in an appropriate host cell as described in Goeddel, Gene Expression Technology: Methods in Enzymology 185, Academic Press, San Diego, Calif (1990). Non-limiting examples of regulatory sequences include: polyadenylation signals, promoters (such as CMV, ASV, SV40, or other viral promoters such as those derived from bovine papilloma, polyoma, and Adenovirus 2 viruses (Fiers, et al., 1973, Nature 273:113; Hager G L, et al., Curr Opin Genet Dev, 2002, 12(2):137-41) enhancers, and other expression control elements. Practitioners in the art understand that designing an expression vector can depend on factors, such as the choice of host cell to be transfected and/or the type and/or amount of desired protein to be expressed.

Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication.

For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest (for example, a modified TFF2 polypeptide) is stably integrated into the genome of eukaryotic cells (for example mammalian cells, such as HEK293 cells), resulting in the stable expression of transfected genes. An exogenous nucleic acid sequence can be introduced into a cell (such as a mammalian cell, either a primary or secondary cell) by homologous recombination as disclosed in U.S. Pat. No. 5,641,670, the contents of which are herein incorporated by reference.

A gene that encodes a selectable marker (for example, resistance to antibiotics or drugs, such as ampicillin, neomycin, G418, and hygromycin) can be introduced into host cells along with the gene of interest in order to identify and select clones that stably express a gene encoding a protein of interest. The gene encoding a selectable marker can be introduced into a host cell on the same plasmid as the gene of interest or can be introduced on a separate plasmid. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired protein molecule (for example, a modified TFF2 polypeptide).

A host cell strain can be chosen for its ability to modulate the expression of the inserted sequences or to process the expressed modified TFF2 polypeptide in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

An exogenous nucleic acid can be introduced into a cell via a variety of techniques known in the art, such as lipofection, microinjection, calcium phosphate or calcium chloride precipitation, DEAE-dextrin-mediated transfection, or electroporation. Electroporation is carried out at approximate voltage and capacitance to result in entry of the DNA construct(s) into cells of interest. Other methods used to transfect cells can also include modified calcium phosphate precipitation, polybrene precipitation, liposome fusion, and receptor-mediated gene delivery.

Animal or mammalian host cells capable of harboring, expressing, and secreting large quantities of a TFF2 peptide of interest into the culture medium for subsequent isolation and/or purification include, but are not limited to, Human Embryonic Kidney 293 cells (HEK-293) (ATCC CRL-1573); Chinese hamster ovary cells (CHO), such as CHO-K1 (ATCC CCL-61), DG44 (Chasin et al., (1986) Som. CellMolec. Genet, 12:555-556; Kolkekar et al., (1997) Biochemistry, 36:10901-10909; and WO 01/92337 A2), dihydrofolate reductase negative CHO cells (CHO/dhfr-, Urlaub et al., (1980) Proc. Natl. Acad. Sci. U.S.A., 77:4216), and dpl2.CHO cells (U.S. Pat. No. 5,721,121); monkey kidney CV1 cells transformed by SV40 (COS cells, COS-7, ATCC CRL-1651); human embryonic kidney cells (e.g., 293 cells, or 293 cells subcloned for growth in suspension culture, Graham et al., (1977) J. Gen. Virol., 36:59); baby hamster kidney cells (BHK, ATCC CCL-10); monkey kidney cells (CV1, ATCC CCL-70); African green monkey kidney cells (VERO-76, ATCC CRL-1587; VERO, ATCC CCL-81); mouse sertoli cells (TM4; Mather (1980) Biol. Reprod., 23:243-251); human cervical carcinoma cells (HELA, ATCC CCL-2); canine kidney cells (MDCK, ATCC CCL-34); human lung cells (W138, ATCC CCL-75); human hepatoma cells (HEP-G2, HB 8065); mouse mammary tumor cells (MMT 060562, ATCC CCL-51); buffalo rat liver cells (BRL 3A, ATCC CRL-1442); TRI cells (Mather (1982) Annals NYAcad. Sci., 383:44-68); MCR 5 cells; FS4 cells. A cell line transformed to produce a modified TFF2 polypeptide can also be an immortalized mammalian cell line of lymphoid origin, which include but are not limited to, a myeloma, hybridoma, trioma or quadroma cell line. The cell line can also comprise a normal lymphoid cell, such as a B cell, which has been immortalized by transformation with a virus, such as the Epstein Barr virus (such as a myeloma cell line or a derivative thereof).

A host cell strain, which modulates the expression of the inserted sequences, or modifies and processes the nucleic acid in a specific fashion desired also may be chosen. Such modifications (for example, glycosylation and other post-translational modifications) and processing (for example, cleavage) of protein products may be important for the function of the protein. Different host cell strains have characteristic and specific mechanisms for the post-translational processing and modification of proteins and gene products. As such, appropriate host systems or cell lines can be chosen to ensure the correct modification and processing of the foreign protein expressed, such as a modified TFF2 polypeptide. Thus, eukaryotic host cells possessing the cellular machinery for proper processing of the primary transcript, glycosylation, and phosphorylation of the gene product may be used. Non-limiting examples of mammalian host cells include HEK-293, 3T3, W138, BT483, Hs578T, CHO, VERY, BHK, Hela, COS, BT20, T47D, NSO (a murine myeloma cell line that does not endogenously produce any immunoglobulin chains), CRL7030, MDCK, 293, HTB2, and HsS78Bst cells.

Various culturing parameters can be used with respect to the host cell being cultured. Appropriate culture conditions for mammalian cells are well known in the art (Cleveland W L, et al., J Immunol Methods, 1983, 56(2): 221-234) or can be determined by the skilled artisan (see, for example, Animal Cell Culture: A Practical Approach 2nd Ed., Rickwood, D. and Hames, B. D., eds. (Oxford University Press: New York, 1992)). Cell culturing conditions can vary according to the type of host cell selected. Commercially available medium can be utilized.

Cells suitable for culturing can contain introduced expression vectors, such as plasmids or viruses. The expression vector constructs can be introduced via transformation, microinjection, transfection, lipofection, electroporation, or infection. The expression vectors can contain coding sequences, or portions thereof, encoding the proteins for expression and production. Expression vectors containing sequences encoding the produced proteins and polypeptides, as well as the appropriate transcriptional and translational control elements, can be generated using methods well known to and practiced by those skilled in the art. These methods include synthetic techniques, in vitro recombinant DNA techniques, and in vivo genetic recombination which are described in J. Sambrook et al., 201, Molecular Cloning, A Laboratory Manual, Cold Spring Harbor Press, Cold Spring Harbor, N.Y. and in F. M. Ausubel et al., 1989, Current Protocols in Molecular Biology, John Wiley & Sons, New York, N.Y. Purification of Recombinant Proteins

Modified TFF2 polypeptide can be purified from any human or non-human cell which expresses the polypeptide, including those which have been transfected with expression constructs that express a modified TFF2 polypeptide. A purified modified TFF2 polypeptide can be separated from other compounds which normally associate with TFF2 such as certain proteins, carbohydrates, or lipids, using methods known in the art. For protein recovery, isolation and/or purification, the cell culture medium or cell lysate is centrifuged to remove particulate cells and cell debris. The desired modified TFF2 polypeptide is isolated or purified away from contaminating soluble proteins and polypeptides by suitable purification techniques. Non-limiting purification methods for proteins include: size exclusion chromatography; affinity chromatography; ion exchange chromatography; ethanol precipitation; reverse phase HPLC; chromatography on a resin, such as silica, or cation exchange resin, e.g., DEAE; chromatofocusing; SDS-PAGE; ammonium sulfate precipitation; gel filtration using, e.g., Sephadex G-75, Sepharose®; protein A Sepharose chromatography for removal of immunoglobulin contaminants; and the like. Other additives, such as protease inhibitors (e.g., PMSF or proteinase K) can be used to inhibit proteolytic degradation during purification. Purification procedures that can select for carbohydrates can also be used, e.g., ion-exchange soft gel chromatography, or HPLC using cation- or anion-exchange resins, in which the more acidic fraction(s) is/are collected.

### EXAMPLES

Examples are provided below to facilitate a more complete understanding of the present disclosure. The following examples illustrate the exemplary modes of making and practicing the invention. However, the scope of the disclosure is not limited to specific embodiments disclosed in these Examples, which are for purposes of illustration only, since alternative methods can be utilized to obtain similar results.

### Example 1-Codon Optimization of Human Modified TFF2 Polypeptide with a his Strep-Tag

Codon Optimized of human modified TFF2 polypeptide with a His_Stre-ta is shown in SE ID NO: 32 below:

| | |
|---|---|
| 1 | ATG GGC AGA AGA GAC GCA CAG CTA TTA GCT GCT CTG CTG GTG |
| | TTA GGA CTG TGT GCT TTG GCT GGA TCT GAG AAG |
| 76 | CCT TCT CCT TGC CAG TGT TCT AGA CTG AGC CCC CAC AAT AGG |
| | ACC AAT TGC GGC TTT CCA GGC ATC ACC TCT GAT |
| 151 | CAG TGC TTC GAT AAT GGC TGC TGC TTC GAT AGC AGC GTT ACA |
| | GGC GTT CCT TGG TGC TTC CAT CCT CTG CCT AAA |
| 226 | CAG GAA AGC GAT CAG TGC GTG ATG GAG GTG TCT GAC AGA |
| | AGG AAT TGC GGC TAT CCT GGC ATC TCT CCT GAA GAA |
| 301 | TGT GCC AGC AGG AAG TGC TGC TTC AGC AAC TTC ATC TTC GAG |
| | GTT CCT TGG TGC TTC TTC CCC AAG TCT GTG GAG |
| 376 | GAC TGC CAC TAC GAG AAC CTG TAC TTT CAA GGA GGA GGA GGA |
| | GGA GGA TCT CAC CAC CAT CAC CAC CAC CAC CAC |
| 451 | CAT CAT GGA GGA GGA GGA TCT GGA GGA TCT TGG TCT CAT CCT |
| | CAG TTT GAG AAG TAG |

The deduced amino acid sequence produced from the optimized DNA sequence is shown below: SEQ ID NO: 33.

| | |
|---|---|
| 1 | MGRRDAQLLA ALLVLGLCAL AGSEKPSPCQ CSRLSPHNRT NCGFPGITSD |
| 51 | QCFDNGCCFD SSVTGVPWCF HPLPKQESDQ CVMEVSDRRN CGYPGISPEE |
| 101 | CASRKCCFSN FIFEVPWCFF PKSVEDCHYE NLYFQGGGGG GSHHHHHHHH |
| 151 | HHGGGGSGGS WSHPQFEK* |

### Example 2—Codon Optimization of Human Modified TFF2-C-Terminal HULGI Fc-Tag polypeptide

Codon optimized DNA Sequence TFF2-C-terminal HULG1 FC-tag SEQ ID NO: 34 is shown below.

| | |
|---|---|
| 1 | ATGGGCAGAA GAGACGCACA GCTATTAGCT GCTCTGCTGG |
| | TGTTAGGACT GTGTGCTTTG GCTGGATCTG AGAAGCCTTC |
| | TCCTTGCCAG TGTTCTAGAC |
| 101 | TGAGCCCCCA CAATAGGACC AATTGCGGCT TTCCAGGCAT |
| | CACCTCTGAT CAGTGCTTCG ATAATGGCTG CTGCTTCGAT |
| | AGCAGCGTTA CAGGCGTTCC |
| 201 | TTGGTGCTTC CATCCTCTGC CTAAACAGGA AAGCGATCAG |
| | TGCGTGATGG AGGTGTCTGA CAGAAGGAAT TGCGGCTATC |
| | CTGGCATCTC TCCTGAAGAA |
| 301 | TGTGCCAGCA GGAAGTGCTG CTTCAGCAAC TTCATCTTCG |
| | AGGTTCCTTG GTGCTTCTTC CCCAAGTCTG TGGAGGACTG |
| | CCACTATGGA GGAGGAGGAT |
| 401 | CTGGAGGATC TGCTAGCACA AAAGGACCTA GCGTTTTTCC |
| | TCTGGCCCCA TCTAGCAAGA GCACATCTGG CGGAACAGCT |
| | GCTTTGGGAT GTCTGGTGAA |
| 501 | GGATTACTTT CCCGAGCCTG TGACAGTGAG CTGGAATTCT |
| | GGAGCCCTGA CATCTGGAGT GCACACCTTT CCTGCTGTTC TGCAGTCTTC |
| | TGGCCTGTAT |
| 601 | TCTCTGTCTA GCGTGGTGAC AGTGCCTAGC TCTTCTCTGG |
| | GAACACAGAC CTACATCTGC AACGTGAACC ACAAGCCCAG |
| | CAACACCAAG GTGGACAAGA |
| 701 | AAGTGGAGCC TAAGAGCTGC GATAAGACCC ACACATGTCC |
| | TCCATGTCCT GCCCCTGAAC TGTTAGGAGG ACCTAGCGTT TTCCTGTTTC |
| | CACCTAAGCC |
| 801 | CAAAGATACC CTGATGATCA GCAGGACCCC TGAGGTGACC |
| | TGTGTGGTGG TTGATGTGAG CCATGAGGAT CCTGAAGTGA |
| | AGTTCAACTG GTACGTGGAT |
| 901 | GGCGTGGAAG TGCACAACGC CAAGACCAAG CCTAGAGAAG |
| | AGCAGTACAA TAGCACCTAC AGAGTGGTGA GCGTGCTGAC |
| | AGTGCTGCAC CAGGATTGGC |
| 1001 | TGAATGGCAA GGAGTATAAG TGCAAGGTGA |
| | GCAATAAGGC CCTGCCAGCC CCTATCGAGA AGACCATCTC |
| | TAAGGCCAAG GGACAACCTA GAGAACCACA |
| 1101 | GGTTTACACA CTGCCCCCCA GCAGAGATGA GCTGACCAAA |
| | AACCAGGTGT CTCTGACATG TCTGGTGAAG GGCTTTTATC |
| | CCAGCGACAT CGCCGTGGAA |
| 1201 | TGGGAGTCTA ATGGACAGCC CGAGAATAAC TACAAGACCA |
| | CACCTCCAGT GCTGGATAGC GATGGCAGCT TCTTCCTGTA |
| | CAGCAAGCTG ACCGTGGATA |
| 1301 | AAAGCAGATG GCAACAGGGC AACGTGTTTA GCTGCAGCGT |
| | GATGCATGAA GCCCTGCACA ACCACTATAC CCAGAAAAGC |
| | CTGAGCCTGT CTCCTGGCAA |
| 1401 | GTAA |

The deduced amino acid sequence produced from the optimized DNA sequence is shown below: SEQ ID NO: 35

| | |
|---|---|
| 1 | MGRRDAQLLA ALLVLGLCAL AGSEKPSPCQ CSRLSPHNRT |
| | NC GFP GIT SD |
| 51 | QCFDNGCCFD SSVTGVPWCF HPLPKQESDQ CVMEVSDRRN |
| | CGYPGISPEE |
| 101 | CASRKCCFSN FIFEVPWCFF PKSVEDCHYG GGGSGGSAST |
| | KGPSVFPLAP |
| 151 | SSKSTSGGTA ALGCLVKDYF PEPVTVSWNS GALTSGVHTF |
| | PAVLQSSGLY |
| 201 | SLSSVVTVPS SSLGTQTYIC NVNHKPSNTK VDKKVEPKSC |
| | DKTHTCPPCP |
| 251 | APELLGGPSV FLFPPKPKDT LMISRTPEVT CVVVDVSHED |
| | PEVKFNWYVD |
| 301 | GVEVHNAKTK PREEQYNSTY RVVSVLTVLH QDWLNGKEYK |
| | CKVSNKALPA |
| 351 | PIEKTISKAK GQPREPQVYT LPPSRDELTK NQVSLTCLVK |
| | GFYPSDIAVE |
| 401 | WESNGQPENN YKTTPPVLDS DGSFFLYSKL TVDKSRWQQG |
| | NVFSCSVMHE |
| 451 | ALHNHYTQKS LSLSPGK* |

### Example 3—Codon Optimization of Human TFF2-HSA

Human TFF2-HSA codon optimized DNA is shown below (SEQ ID NO: 36).

>Human TFF2-HSA_ Codon Optimized DNA

| | |
|---|---|
| 1 | ATGGGCAGAA GAGACGCACA GCTATTAGCT GCTCTGCTGG |
| | TGTTAGGACT GTGTGCTTTG GCTGGATCTG AGAAGCCTTC |
| | TCCTTGCCAG TGTTCTAGAC |
| 101 | TGAGCCCCCA CAATAGGACC AATTGCGGCT TTCCAGGCAT |
| | CACCTCTGAT CAGTGCTTCG ATAATGGCTG CTGCTTCGAT |
| | AGCAGCGTTA CAGGCGTTCC |
| 201 | TTGGTGCTTC CATCCTCTGC CTAAACAGGA AAGCGATCAG |
| | TGCGTGATGG AGGTGTCTGA CAGAAGGAAT TGCGGCTATC |
| | CTGGCATCTC TCCTGAAGAA |
| 301 | TGTGCCAGCA GGAAGTGCTG CTTCAGCAAC TTCATCTTCG |
| | AGGTTCCTTG GTGCTTCTTC CCCAAGTCTG TGGAGGACTG |
| | CCACTATGGA GGAGGAGGAT |
| 401 | CTGATGCCCA TAAATCTGAA GTGGCCCACA GGTTCAAGGA |
| | TCTGGGAGAG GAGAACTTCA AGGCCCTGGT GCTGATCGCT |
| | TTTGCTCAAT ACCTGCAGCA |
| 501 | GTGCCCTTTT GAGGATCACG TGAAACTGGT GAACGAGGTG |
| | ACCGAGTTTG CCAAGACATG TGTGGCCGAT GAGTCTGCCG |
| | AGAATTGCGA TAAAAGCCTG |
| 601 | CACACCCTGT TCGGAGACAA GCTGTGTACA GTGGCTACCC |
| | TGAGAGAGAC ATATGGCGAA ATGGCCGATT GTTGCGCCAA |
| | ACAGGAACCC GAGAGAAATG |
| 701 | AGTGCTTCCT GCAGCACAAG GACGACAACC CTAATCTGCC |
| | TAGGCTGGTT AGACCTGAGG TGGATGTGAT GTGTACCGCC |
| | TTCCACGACA ATGAGGAGAC |
| 801 | ATTCCTGAAG AAGTACCTGT ACGAGATCGC CCGGAGACAC |
| | CCTTACTTCT ACGCCCCTGA ACTGCTGTTT TTCGCCAAGA |
| | GATACAAAGC CGCCTTTACC |
| 901 | GAGTGCTGTC AGGCTGCCGA TAAAGCTGCC TGTTTACTGC |
| | CCAAGCTGGA TGAACTGAGA GATGAGGGAA AGGCCTCTAG |
| | CGCCAAGCAG AGACTGAAAT |
| 1001 | GTGCTAGCCT GCAGAAGTTT GGCGAAAGAG |
| | CCTTTAAAGC CTGGGCTGTG GCCAGACTGA GCCAGAGATT |
| | TCCTAAAGCC GAGTTTGCCG AAGTGAGCAA |
| 1101 | ATTAGTGACC GACCTGACCA AGGTGCACAC |
| | CGAGTGTTGT CATGGCGATC TTCTGGAATG CGCCGATGAT |
| | AGAGCTGATC TGGCCAAGTA CATCTGCGAG |
| 1201 | AACCAGGATA GCATCAGCAG CAAGCTGAAG |
| | GAGTGTTGCG AGAAACCTCT GCTGGAGAAA AGCCACTGTA |
| | TCGCCGAAGT GGAGAACGAC GAGATGCCTG |
| 1301 | CTGATCTGCC TTCTTTAGCC GCCGATTTTG |
| | TGGAGAGCAA GGATGTGTGC AAGAACTACG CCGAGGCCAA |
| | AGACGTGTTT TTGGGCATGT TCCTGTACGA |
| 1401 | GTACGCCAGA AGACACCCTG ATTATAGCGT |
| | GGTGCTGCTG CTGAGACTGG CCAAGACATA CGAGACAACA |
| | CTGGAGAAGT GTTGTGCTGC TGCTGATCCT |
| 1501 | CACGAGTGTT ACGCCAAGGT GTTCGACGAG |
| | TTCAAACCTC TGGTGGAAGA ACCTCAGAAC CTGATCAAGC |
| | AGAACTGCGA GCTGTTCGAG CAGCTGGGCG |
| 1601 | AGTACAAGTT CCAGAATGCT CTGCTGGTGA |
| | GATACACCAA GAAAGTGCCT CAGGTGTCTA CCCCCACCCT |
| | GGTTGAAGTG AGCAGAAATC TGGGCAAAGT |
| 1701 | GGGCTCTAAA TGTTGCAAGC ACCCTGAGGC |
| | CAAGAGGATG CCTTGTGCCG AGGATTATCT GTCTGTGGTG |
| | CTGAATCAAC TGTGTGTGCT GCACGAGAAG |
| 1801 | ACCCCTGTGA GCGACAGAGT GACAAAGTGT |
| | TGTACCGAGT CTCTGGTGAA CAGAAGACCC TGCTTTTCTG |
| | CCCTGGAGGT GGATGAGACC TATGTGCCTA |
| 1901 | AGGAGTTCAA TGCCGAGACC TTTACCTTCC |
| | ATGCCGACAT CTGCACCCTG AGCGAGAAAG AGAGGCAGAT |
| | CAAGAAACAG ACAGCCCTGG TTGAACTGGT |
| 2001 | GAAGCACAAG CCTAAGGCCA CCAAAGAGCA |
| | GCTGAAAGCC GTTATGGACG ATTTTGCCGC CTTTGTGGAG |
| | AAGTGCTGTA AGGCCGACGA TAAGGAGACC |
| 2101 | TGTTTCGCCG AAGAGGGAAA AAAGCTGGTT |
| | GCTGCCTCTC AAGCTGCTCT GGGCCTGTAA TAA |

The deduced TFF2-HSA amino acid sequence is shown below 50 SEQ ID NO: 37:

| | |
|---|---|
| 1 | MGRRDAQLLA ALLVLGLCAL AGSEKPSPCQ CSRLSPHNRT |
| | NCGFPGITSD |
| 51 | QCFDNGCCFD SSVTGVPWCF HPLPKQESDQ CVMEVSDRRN |
| | CGYPGISPEE |
| 101 | CASRKCCFSN FIFEVPWCFF PKSVEDCHYG GGGSDAHKSE |
| | VAHRFKDLGE |
| 151 | ENFKALVLIA FAQYLQQCPF EDHVKLVNEV TEFAKTCVAD |
| | ESAENCDKSL |
| 201 | HTLFGDKLCT VATLRETYGE MADCCAKQEP ERNECFLQHK |
| | DDNPNLPRLV |
| 251 | RPEVDVMCTA FHDNEETFLK KYLYEIARRH PYFYAPELLF |
| | FAKRYKAAFT |
| 301 | ECCQAADKAA CLLPKLDELR DEGKASSAKQ RLKCASLQKF |
| | GERAFKAWAV |
| 351 | ARLSQRFPKA EFAEVSKLVT DLTKVHTECC HGDLLECADD |
| | RADLAKYICE |
| 401 | NQDSISSKLK ECCEKPLLEK SHCIAEVEND EMPADLPSLA |
| | ADFVESKDVC |
| 451 | KNYAEAKDVF LGMFLYEYAR RHPDYSVVLL LRLAKTYETT |
| | LEKCCAAADP |
| 501 | HECYAKVFDE FKPLVEEPQN LIKQNCELFE QLGEYKFQNA |
| | LLVRYTKKVP |
| 551 | QVSTPTLVEV SRNLGKVGSK CCKHPEAKRM PCAEDYLSVV |
| | LNQLCVLHEK |
| 601 | TPVSDRVTKC CTESLVNRRP CFSALEVDET YVPKEFNAET |
| | FTFHADICTL |
| 651 | SEKERQIKKQ TALVELVKHK PKATKEQLKA VMDDFAAFVE |
| | KCCKADDKET |
| 701 | CFAEEGKKLV AASQAALGL** |

### Example 4—Codon Optimization of Human TFF2-CTPX2-FLAG X3

Human TFF2-CTPX2-FLAG X3_Codon Optimized DNA (SEQ ID NO: 38)

| | |
|---|---|
| 1 | ATGGGCAGAA GAGACGCACA GCTATTAGCT GCTCTGCTGG |
| | TGTTAGGACT GTGTGCTTTG GCTGGATCTG AGAAGCCTTC |
| | TCCTTGCCAG TGTTCTAGAC |
| 101 | TGAGCCCCCA CAATAGGACC AATTGCGGCT TTCCAGGCAT |
| | CACCTCTGAT CAGTGCTTCG ATAATGGCTG CTGCTTCGAT |
| | AGCAGCGTTA CAGGCGTTCC |
| 201 | TTGGTGCTTC CATCCTCTGC CTAAACAGGA AAGCGATCAG |
| | TGCGTGATGG AGGTGTCTGA CAGAAGGAAT TGCGGCTATC |
| | CTGGCATCTC TCCTGAAGAA |
| 301 | TGTGCCAGCA GGAAGTGCTG CTTCAGCAAC TTCATCTTCG |
| | AGGTTCCTTG GTGCTTCTTC CCCAAGTCTG TGGAGGACTG |
| | CCACTACAGC AGCTCTTCTA |
| 401 | AAGCTCCTCC TCCTTCTCTG CCTTCTCCTT CTAGACTTCC |
| | TGGCCCTAGC GATACCCCTA TTCTGCCTCA AAGCAGCAGC |
| | TCTAAAGCTC CTCCTCCTTC |
| 501 | TTTACCTAGC CCCAGCAGAC TTCCTGGACC TTCTGATACC |
| | CCTATCCTGC CTCAAACAGG CATGGACTAT AAGGACGACG |
| | ACGACAAGGA CTACAAGGAC |
| 601 | GACGACGACA AGGACTACAA GGATGACGAC |
| | GACAAAGCCA GCTAA |

The deduced TFF2-HSA amino acid sequence is shown below (SEQ ID NO: 39)

| | |
|---|---|
| 1 | MGRRDAQLLA ALLVLGLCAL AGSEKPSPCQ CSRLSPHNRT |
| | NCGFPGITSD |
| 51 | QCFDNGCCFD SSVTGVPWCF HPLPKQESDQ CVMEVSDRRN |
| | CGYPGISPEE |
| 101 | CASRKCCFSN FIFEVPWCFF PKSVEDCHYS SSSKAPPPSL |
| | PSPSRLPGPS |
| 151 | DTPILPQSSS SKAPPPSLPS PSRLPGPSDT PILPQTGMDY |
| | KDDDDKDYKD |
| 201 | DDDKDYKDDD DKAS* |

All constructs of Examples 1-4 are expressed in a CHO-S transient system. Expression of the three variants are analyzed for expression using Western Blot and anti-huTFF2.

### Example 5—Measurement of Modified TFF2 Polypeptide Activity by Calcium Mobilization

Jurkat cells, KATO-III and/or AsPC-1 cells (2.5×10⁶ cells/ml) are resuspended in RPMI 1640 medium containing 0.5% BSA and incubated with the Ca²⁺-binding dye Indo-1 AM at a final concentration of 5 mM for 1 hr at 37° C. in the dark with agitation. Loaded cells are washed, resuspended in Hanks' balanced salt solution medium containing 2 mM CaCl₂) and 1 mM MgCl₂, and left for 20 min at room temperature. Cells are aliquoted into fluorescence-activated cell sorter tubes that are immediately transferred into a 37° C. water bath for an additional 5 min prior to measurements. Equilibrated cells are then used for flow cytometric analysis of the Ca²+level using an LSRII machine (BD Biosciences). The base-line intracellular Ca²⁺level is recorded for an initial 25-30 s followed by a stimulation with the indicated concentrations of SDF-la, TFF2, gastrin, ionomycin, or diluent (phosphate-buffered saline). Data collection is continued at the speed of 2000 events/s for an additional 4-10 min. An increase in binding of cytosolic Ca²⁺to Indo-1 results in a change of the emission spectrum of Indo-1 from 510 nm (free form) to 420 nm (Ca²⁺-bound form). Thus, blue (4′,6-diamidino-2-phenyl-indole channel, 420 nm) and violet (Indo channel, 510 nm) cell fluorescence are measured, and data is plotted using FlowJo software (version 6.4; Tree Star, Inc.). Intracellular calcium mobilization in response to SDF-la 25 or TFF2 in the presence of AMD3100 or anti-CXCR4 antibody is measured after cells are preincubated for 40 min at 37° C. with AMD3100 or with anti-CXCR4 mAbs 12G5 or 2B11 (eBioscience) accordingly.

### Example 6-Measurement of Modified TFF2 Polypeptide Activity by Phosphorylation of ERK1 2

Measurement of the activity modified TFF2 polypeptide is performed by phosphorylation of ERK1/ERK2 in Jurkat human acute T cell leukemic cells, KATO-III human stomach cancer cells, and/or AsPC-1 human pancreatic cells (all cell lines provided by ATCC) by using the AlphaLISA SureFire Ultra p-ERK ½ (Thr202/Tyr204) assay kit by Perkin Elmer. Cell lines are thawed and expanded according to the instructions provided by ATCC. Cells are harvested by centrifugation and resuspended in HBSS at a 10⁷ cells/mL. Cells are seeded at 4 mL of cells/well into 384-well while opaque culture plate (PerkinElmer) and incubated at 37° C. for 1-2 hours. Wild-type and variants of recombinant TFF2 in 4 mL at a concentration of 10-30 mg/mL in HBSS containing 0.1% BSA are added to the plates to stimulate the cells and incubated at 37° C. for 5-30 minutes. Cells are lysed with 2 mL/well lysis buffer, followed by the addition of 5 mL Acceptor Mix. Plates are then sealed with Topseal-A adhesive film and incubated for 1 hr at room temperature. 5 mL Donor Mix and then added to the wells under subdued light, sealed with Topseal-A adhesive film, covered with foil and incubated for 1 hr at room temperature in the dark. Plates are read on a AlphaPlex compatable plate reader using standard AlphaPlex settings. Inhibition of TFF2 stimulation of CXCR4 is performed with AMD3100 (Sigma), a small molecule antagonist of CXCR4, or the anti-CXCR4 mAbs 12G5 and 2B11 (eBioscience) for 1-2 hours at 37° C. before the addition of recombinant TFF2.

### Example 7—Colorectal Adenocarcinoma

A 51-year old male presented without a family history of early onset colorectal cancer or other malignancies potentially consistent with a Lynch syndrome kindred, who was in his usual state of health until he underwent his first routine screening evaluation by colonoscopy and is found to have a partially obstructing mass in the transverse colon. Biopsy confirms the presence of a moderately-differentiated adenocarcinoma with lymphovascular invasion. Reflexive molecular testing is notable for KRAS exon 2 mutation: (+), BRAF mutation: (−). However, the patient is identified as metastatic microsatellite instability-high (MSI-H) or mismatch repair deficient (dMMR) or dMMVIR/MSI-H: (+). Subsequent staging CT scans identified both small volume hepatic and peritoneal disease without extra-abdominal metastases. The patient is classified as having asymptomatic stage IV colorectal cancer and successfully completes a transverse colectomy without difficulty. Six weeks thereafter, the patient is started on a regimen of levofolinic acid (FOL or Fusilev®), 5-fluorouracil (5-FU or F) and oxaliplatin (OX or Eloxatin®, a platinum cytotoxic agent that forms both inter- and intra-strand cross links in DNA) or “FOLFOX” plus bevacizumab (Avastin®). Restaging after cycle #6 of FOLFOX-bevacizumab is consistent with a near complete remission (nCR). Despite tolerating therapy well, the patient's health declines continuing “maintenance” bevacizumab/fluoropyrimidine and he enters an observation program. The patient does well for 14 months at which time he notes the onset of dyspnea. CT scans notable for a new large right pleural effusion, ascites and progressive hepatic metastases with significant liver function test abnormalities. Large volume thoracentesis confirms the presence of a malignant pleural effusion with cytologic evidence of adenocarcinoma. The patient undergoes placement of a chest tube followed by successful pleurodesis. The patient starts second line therapy with FOLFIRI-bevacizumab. The patient again tolerates therapy well and restaging CT scans after cycle #4 are consistent with a partial remission (PR). The patient continues levofolinic acid (FOL), 5-FU (F) and irinotecan (IRI or Camptosar®, an inhibitor of topoisomerase I) FOLFIRI-bevacizumab with plans to treat to progression as allowed by toxicity. Restaging after cycle #10 of FOLFIRI-Avastin documents progressive liver metastases and recurrent ascites. The patient is initiated on therapy with single agent pembrolizumab (Keytruda®) but does not respond. Subsequently therapy is initiated with modified TFF2 polypeptides and the patient achieves a partial objective response. Combined treatment is initiated with modified TFF2 polypeptides and pembrolizumab, which results in a complete response and in regression of tumors and metastasis.

### Example 9-Esophageal Squamous Cell Carcinoma

The patient is a 58-year old male with a history of tobacco abuse who is in his usual state of health until he presents with dysphagia and intermittent subxiphoid discomfort. After failing several palliative interventions including both histamine receptor-type 2 (H2) blockers and proton pump blockers, he is seen in formal gastroenterology consultation and undergoes an upper endoscopy at which time he is informed of a partially obstructing, 3.2 cm, exophytic, mid-esophageal mass. Review of the pathology reveals a moderately differentiated squamous cell carcinoma. PD-L1 combined positive score (CPS):20%. Staging PET/CT scans and endoscopic ultrasound are consistent with T4aNO disease and confirms that the tumor is amenable to resection. The patient successfully completes concurrent low-dose weekly neoadjuvant carboplatinum/paclitaxel (a tubulin inhibitor, Taxol®) and radiotherapy followed by definitive surgical resection. Review of the surgical pathology fails to reveal any evidence of residual malignancy. Nine months later, the patient presents with anorexia and weight loss. CT scans documented the presence of both hepatic and pulmonary metastases. CT-guided core needle biopsy confirms the presence of metastatic squamous cell carcinoma. PD-L1 combined positive score (CPS):16%.Since the progression free survival (PFS) is >six months after first line chemotherapy (in this case neoadjuvant) and the patient has a good performance status (ECOG <1), and particularly if the patient presents with either rapidly progressive or highly symptomatic disease that requires an early and meaningful response just to stabilize the situation, then second line therapy is initiated. The patient receives six cycles of FOLFIRI and achieves a plateau phase partial remission (PR) and enters onto an observation program. Four months later, routine surveillance CT scans show progression of metastatic disease. The patient maintains an ECOG 1 performance status and wishes to pursue additional therapy. The patient is initiated on single agent pembrolizumab but does not respond. Subsequently, therapy is initiated with modified TFF2 polypeptides and the patient achieves a partial objective response. Combined treatment is initiated with modified TFF2 polypeptides and pembrolizumab, which results in a complete response and in regression of tumors and metastasis.

### Example 10-Gastric Esophageal Adenocarcinomas

The patient is a 47-year old female who is in her usual state of health until she presented 18 months ago with dyspepsia and intermittent subxiphoid discomfort. After failing several palliative interventions including both H2 and proton pump blockers, she is seen in formal gastroenterology consultation and undergoes an upper endoscopy at which time she is informed of a 2.2 cm exophytic mass in (gastric cardia/distal esophagus). Review of the pathology reveals a poorly differentiated adenocarcinoma. There is no evidence of an H. pylori infection and Her2 immunohistochemical (IHC) staining was 0. PD-L1 combined positive score (CPS):12%. Staging CT scans document the presence of both regional lymphadenopathy and low volume hepatic metastases. The patient is classified as having an unresectable, low volume stage IV poorly differentiated gastric/esophageal adenocarcinoma. Based on the low volume disease with minimal symptoms and CPS >10, the patient starts single agent pembrolizumab as first line therapy based on the findings of the KEYNOTE-062 trial in which patients with CPS >10, OS (vs. CDDP/fluoropyrimidine) improved (17.4 months vs. 10.8 months) with few all grade or grade ¾ toxicities. However, the patient's condition progresses, and she develops a bulky and symptomatic tumor (6.0 cm primary, extensive liver metastases) and a PD-L1 CPS <10. Subsequently, she receives five cycles of FOLFOX and achieves a plateau phase partial remission (PR). Although therapy is generally well tolerated, she experiences grade 2 peripheral neuropathy. The patient is placed into an observation program. Seven months later, routine surveillance CT scans reveal progressive hepatic metastases as well as a new lung metastasis. The patient maintains an ECOG 1 performance status and treatment was initiated with ramucirmab (Cyramza®, a direct VEGFR2 antagonist) and paclitaxel. Restaging CT scans after cycle #4 are consistent with stable disease. However, the patient's peripheral neuropathy worsenes, and paclitaxel was discontinued. She is maintained single agent ramucirumab but progresses three months later. Based on the results of the KEYNOTE-059 study (failed two or more lines of chemotherapy), the patient is converted to single agent pembrolizumab. The patient is initiated on single agent pembrolizumab but does not respond. Subsequently, therapy is initiated with modified TFF2 polypeptides and the patient achieves a partial objective response. Combined treatment is initiated with modified TFF2 polypeptides and pembrolizumab, which result in a complete response and in regression of tumors and metastasis.

### Example 11-Pancreatic cancer

The patient is a 39-year old female in generally excellent health who is well until she reports the onset of vague mid-thoracic back pain that is controlled with the intermittent use of non-steroidal anti-inflammatory drugs (NSAIDs). The patient presents with night sweats and one week of scleral icterus and darkening urine. Clinical evaluation confirms the presence of jaundice and chemistries identify a pattern of cholestatic liver dysfunction with a total bilirubin of 12.2 mg/dl. CT scans reveal an 8.4 cm mass at the head of the pancreas as well as porta hepatis lymphadenopathy, scattered small, bilateral hepatic masses and significant dilation of the common bile duct. Endoscopic retrograde cholangiopancreatography (ERCP) with hepatic stent placement is successful and the bilirubin returns to normal levels. CT-guided hepatic biopsy confirms the presence of a poorly differentiated KRAS: (+) TP53: (+) adenocarcinoma. CA 19-9 is markedly elevated (710). The presentation is most consistent with unresectable stage IV adenocarcinoma of the pancreas. There is no family history of pancreatic, breast or ovarian cancer or a known BRCA2 mutation. The patient undergoes next generation sequencing (NGS). There is no evidence of germline mutations for either BRCA2 or PALB2. However, the patient is dMMR/MSI-H. The patient is initiated on a modified-FOLFIRINOX regimen (FOL+F+irinotecan or “IRIN”+OX) and successfully completes six cycles of therapy that is generally well tolerated. Restaging CT scans after cycles #4 and #6 are consistent with a stable, plateau-phase partial remission. The patient enters onto an observation program and remains well until four months later when routine surveillance CT scans confirms the presence of asymptomatic, low volume progression of hepatic metastases. The patient is initiated on single agent nivolumab (Opdivo®) but does not respond. Subsequently therapy is initiated with modified TFF2 polypeptides and the patient achieves a partial objective response. Combined treatment is initiated with modified TFF2 polypeptides and nivolumab, which results in a complete remission and in regression of tumors.

### Example 12—Stabilized Recombinant TFF2 (TFF2-CTP) Enhances Anti-Tumor Activity of PD-1 Blockade in Mouse Models of Colorectal Cancer

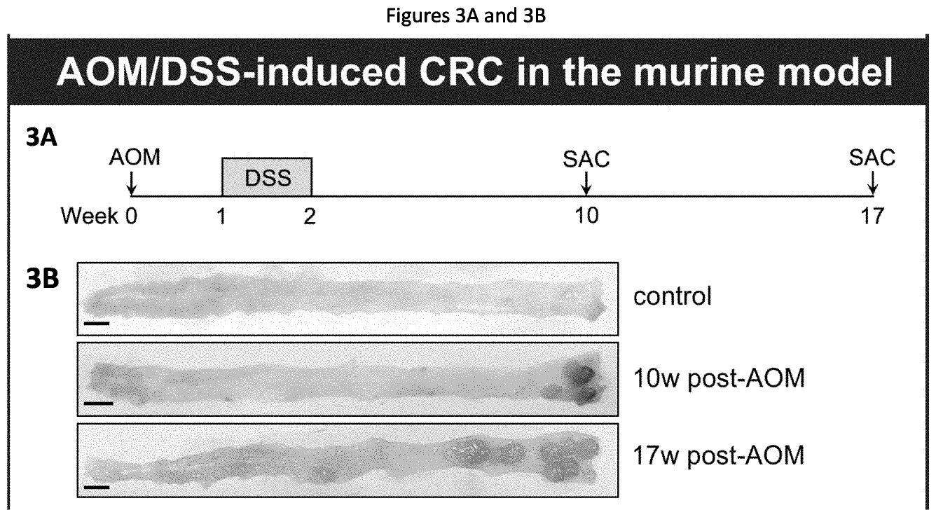

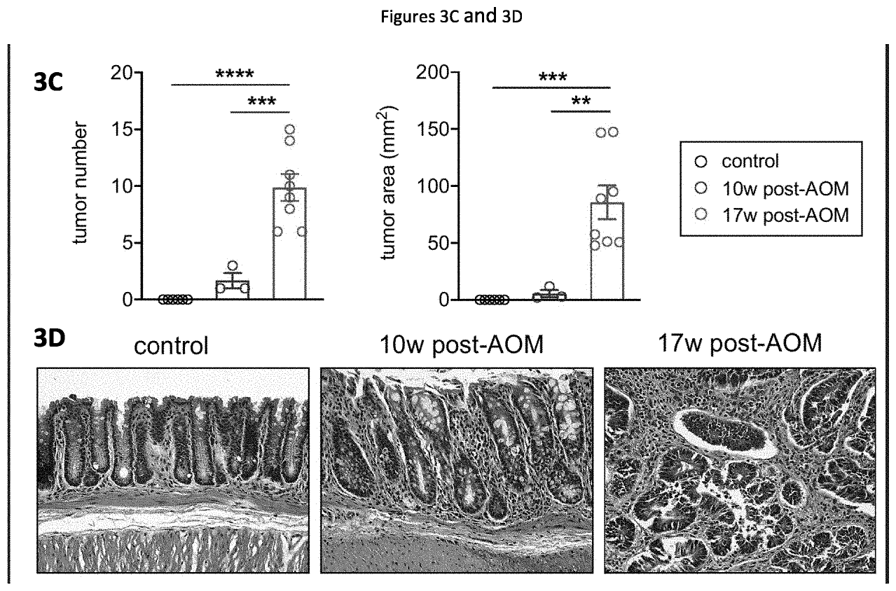

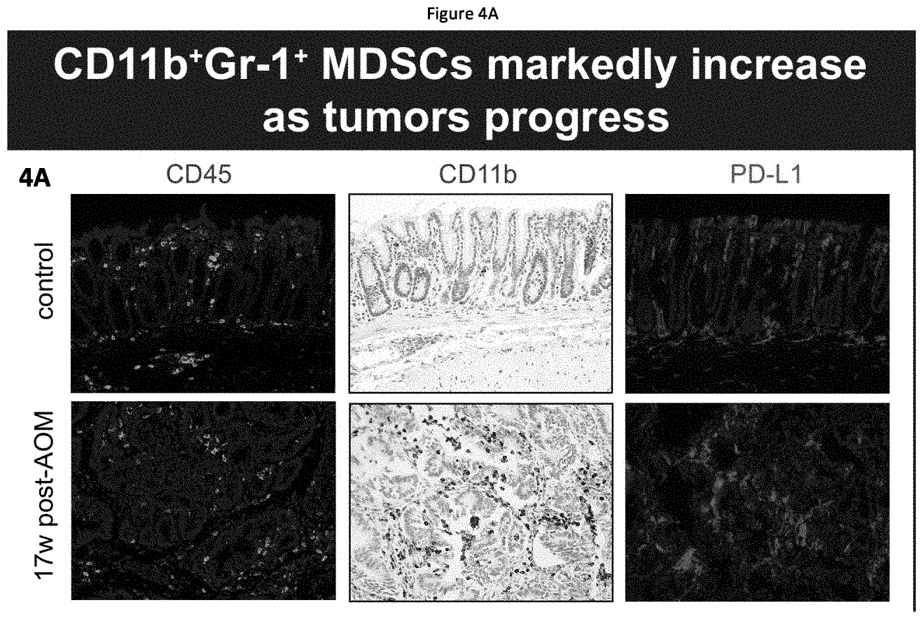

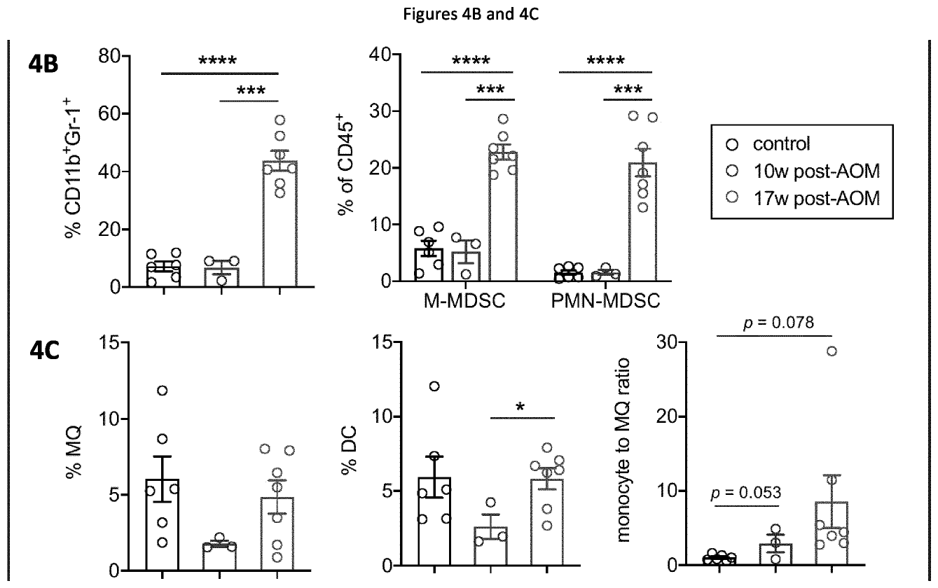

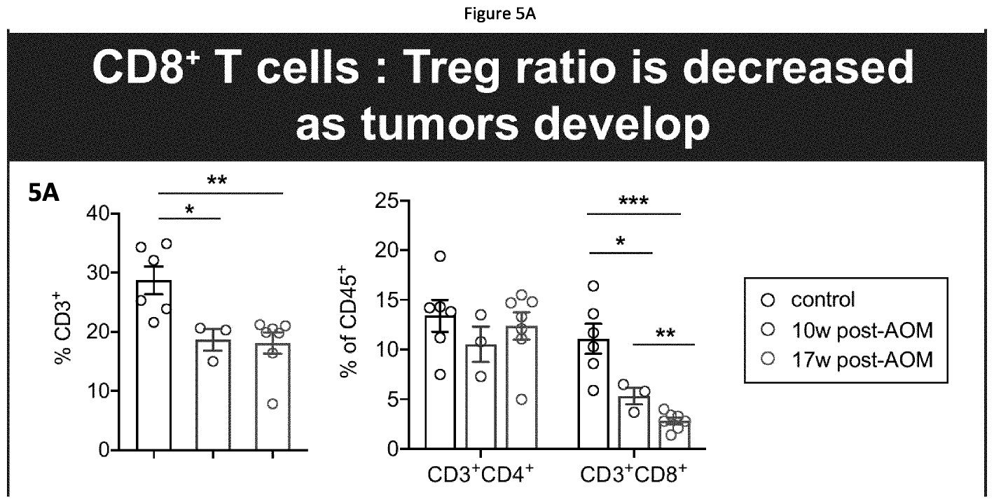

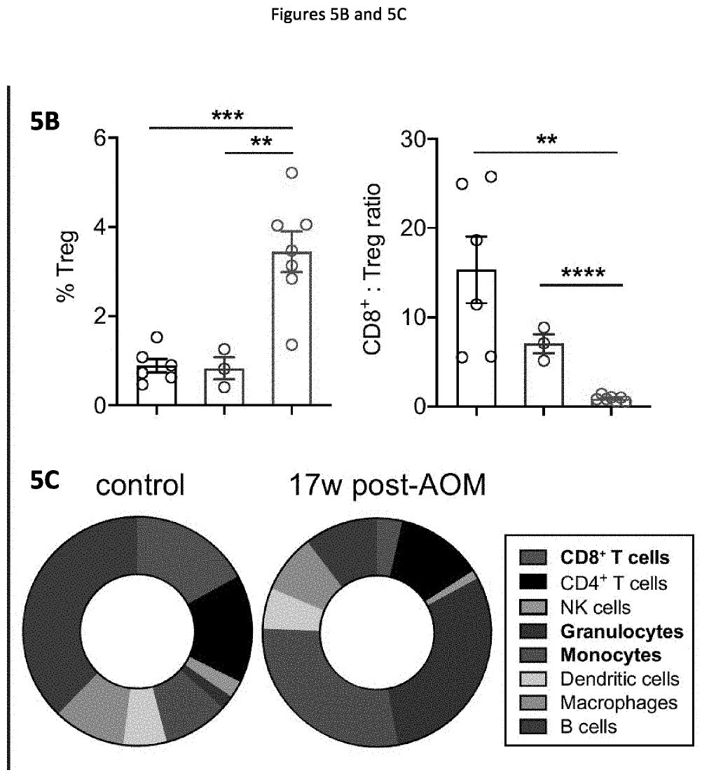

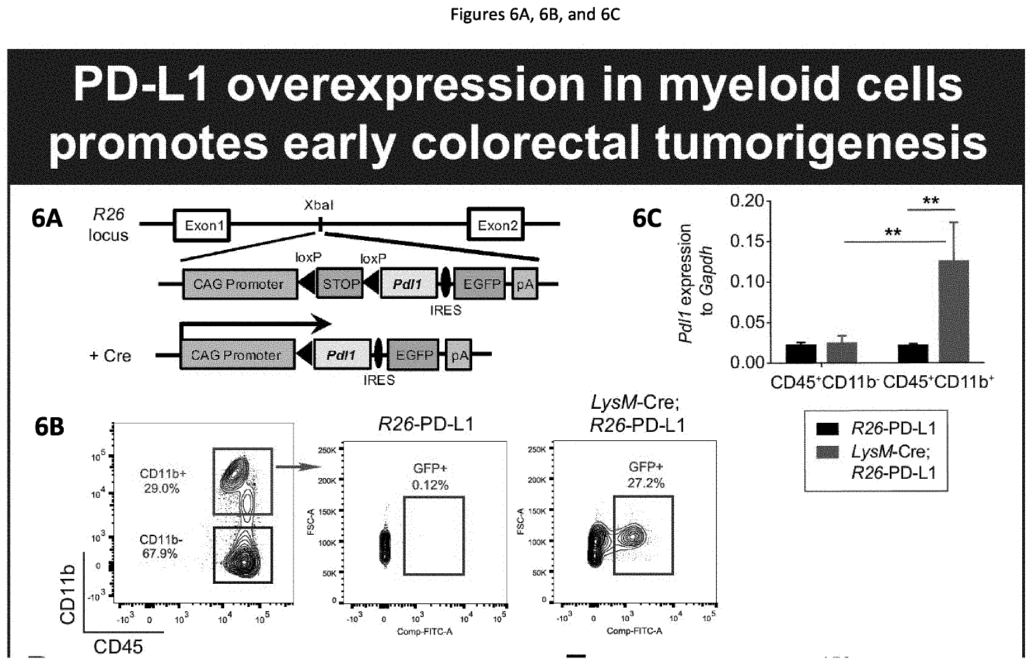

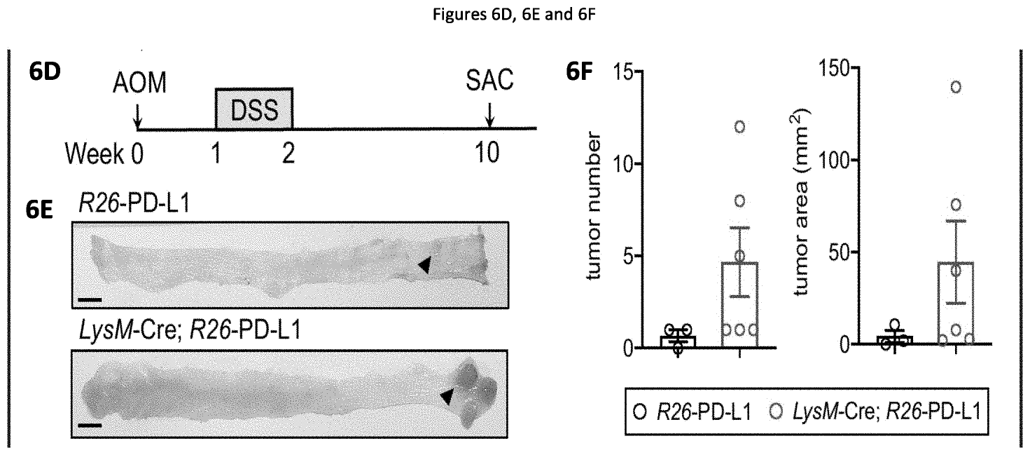

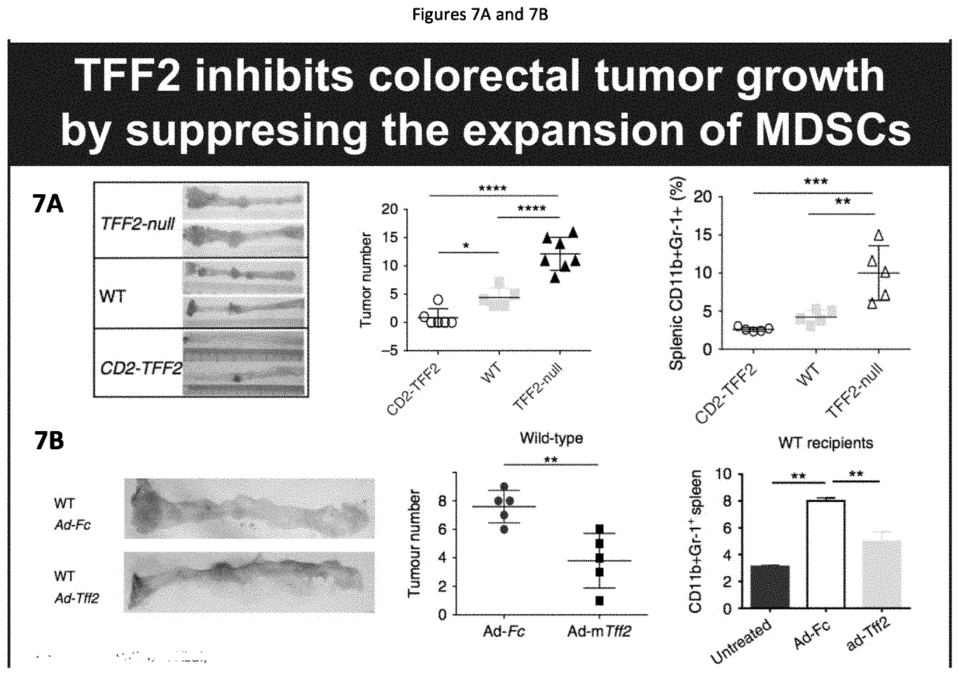

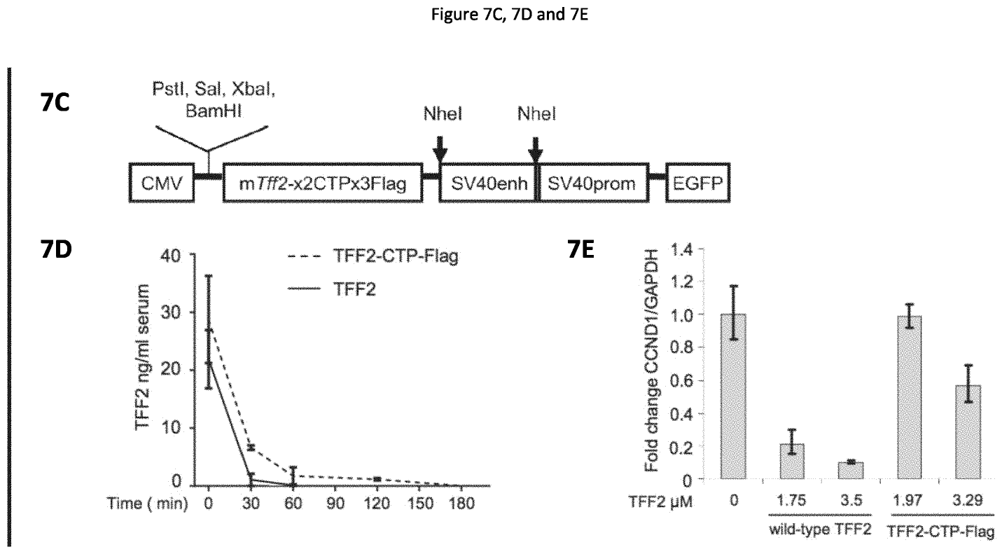

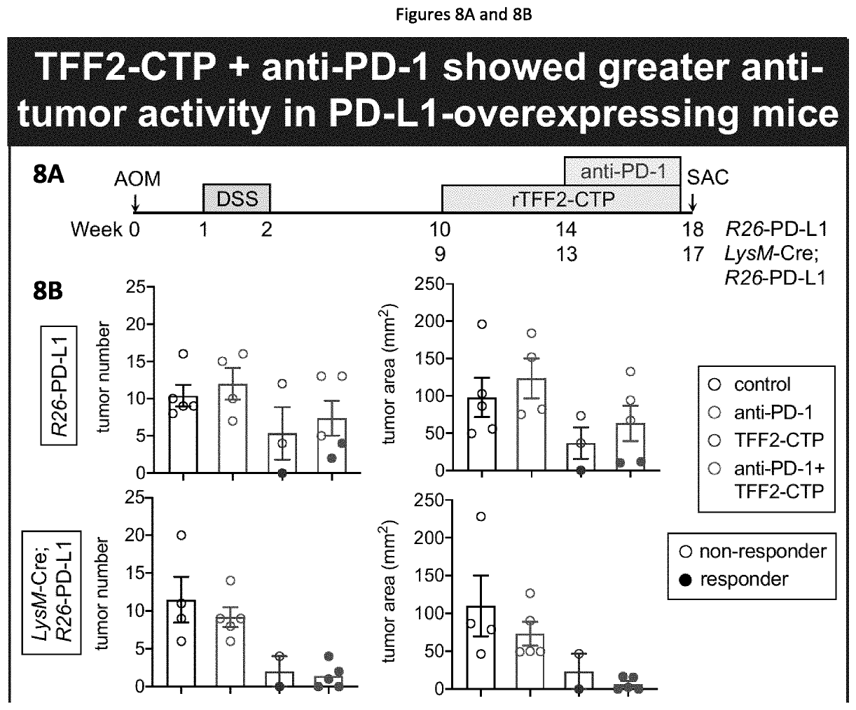

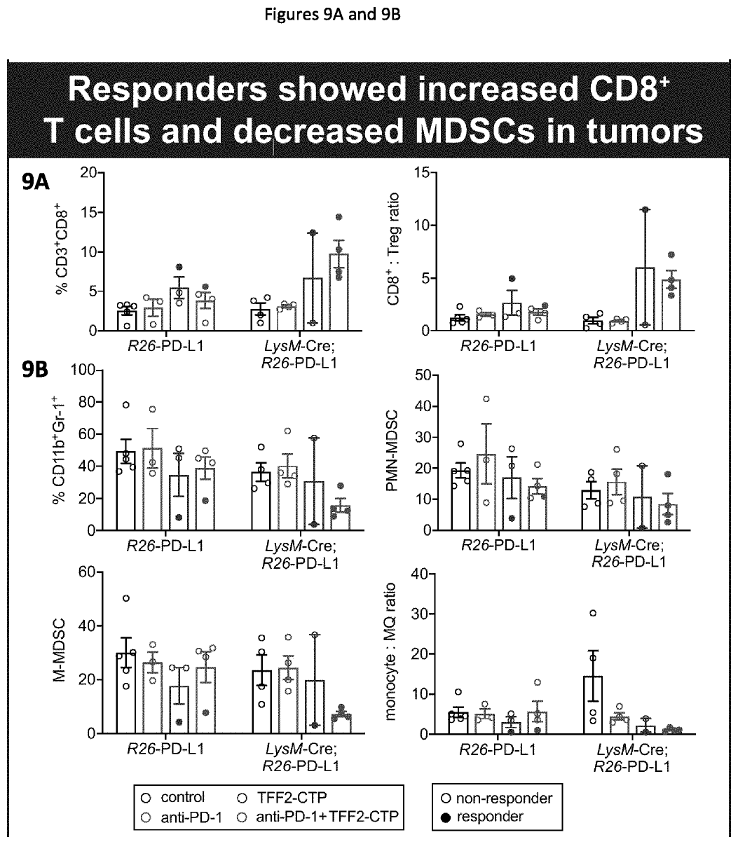

Despite remarkable responses to immune checkpoint blockade across multiple tumor types, the clinical benefit in colorectal cancer (CRC) is limited to microsatellite unstable tumors. PD-L1 expression is a negative prognostic marker in CRC but correlates with a better response to PD-1 blockade. In this Example, the role of PD-L1 in colorectal tumorigenesis was investigated and the utility of targeting myeloid-derived suppressor cells (MDSCs) in combination with PD-1 blockade was evaluated in mouse models of Colorectal cancer (CRC). Knock-in mice that conditionally express the murine Pdll gene (R26-LSL-Pdll-EGFP) were generated and crossed with LysM-Cre mice to overexpress PD-L1 specifically in the myeloid lineage. AOM/DSS-treated mice formed tumors at 10 weeks and developed adenocarcinoma at 17 weeks post-AOM. See FIGS. 3A to 3D. AOM/DSS treatment led to a significant expansion of myeloid cells, particularly CD11b+Gr-1+MDSCs, compared to untreated mice. See FIGS. 4A to 4C. Furthermore, there was a significant decrease in intratumoral CD8+T cells, indicating attenuated anti-tumor immunity. See FIGS. 5A to 5C. AOM/DSS-treated PD-L1-overexpressing LysM-Cre; R26-PD-L1 mice showed markedly enhanced early colorectal tumorigenesis, with a significant increase in tumor number and size. See FIGS. 6A to 6F. TFF2, a secreted anti-inflammatory peptide, inhibits colon tumor growth by suppressing the expansion of CD11b+Gr-1+MDSCs. TFF2 fused with two carboxyl-terminal peptide and three Flag motifs (TFF2-CTP-Flag) prolonged the circulation time in blood but retained bioactivity. See FIGS. 7A to 7E. We induced tumors in R26-PD-L1 and LysM-Cre; R26-PD-L1 mice with AOM/DSS, administered fusion recombinant TFF2-CTP-Flag and/or anti-PD-1 antibody. Anti-PD-1 antibody in combination with TFF2-CTP showed a marked reduction in tumor growth while anti-PD-1 monotherapy failed to suppress growth. Interestingly, combined treatment showed greater anti-tumor activity in PD-L1-overexpressing mice than control animals. See FIG. 8. Treatment responders showed significantly increased tumor-infiltrating CD8+T cells and concomitantly decreased CD1 1b+Gr-1+myeloid cells. See FIG. 9. These early findings suggest that TFF2 augments the response rate of CRC to PD-1 blockade, possibly through suppressing MDSC expansion, supporting the potential of TFF2-CTP in combination I-O treatment for CRC.

Therefore, anti-PD-1 monotherapy was unable to evoke anti-tumor immunity in CRC, but TFF2-CTP augmented the efficacy of anti-PD-1 therapy. Anti-PD-1 in combination with TFF2-CTP showed greater anti-tumor activity in PD-L1-overexpressing mice. Responders to TFF2-CTP alone or in combination with PD-1 blockade had increased tumor-infiltrating CD8+T cells, along with decreased MDSCs.

### Example 13—Expression and Purification of TFF2-Human Serum Albumin (HSA)Fusion

Gene Synthesis

The TFF-2 HSA proteins were codon optimized and synthesized using Codex gene synthesis. The TFF-2 HSA proteins synthesized were: TFF2-HSA [WT]; TFF2-HSA [D I/I]; TFF2-HSA [D II/I]; TFF2-HSA [D II/II]; TFF2-HSA [LBD I/I]; TFF2-HSA [LBD II/I] and TFF2-HSA [LBD II/II]. The oligonucleotides were synthesized by Codex and the genes were assembled in SGI/Codex Assembler. The synthesized genes were subcloned into expression vector pAB2 (digested with XbaI and BamHI) using the SGI. Overlapping 30 bp sequences were used to Gibson assemble the gene of interest into pAB2. The vector with the gene of interest was transformed into NEB® 5-alpha Competent E. coli [(High Efficiency);NEB; C2987H]. Three colonies were picked and scaled up for DNA isolation via mini-prep. The 3 colonies were then sent for sequencing. Upon sequence verification, positive clones were scaled up and plasmid DNA was isolated.

Transfection

On the day before transfection, HEK293 cells were seeded in flasks. On the day of transfection, cell count and culture viability were measured and once the culture reached 1.8×10⁶-2.2×10⁶ cells/mL with a viability of >96%, transfection proceeded. DNA was then resuspended in FectoPro (Polyplus) transfection reagent and diluted in serum free medium and incubated at room temperature. The transfection complex was then added to the HEK293 cells gently while swirling the flask, and subsequently moved back into the 37° C. incubator. The cell cultures were then fed with fresh media 4-5 hours post-transfection. Cell supernatants were harvested, clarified by centrifugation 6 days post-transfection.

Protein Purification

The HSA-tagged human TFF2 proteins were purified with AlbuPure® (product code 3151, Prometic Bioseparations®, Ltd) selective affinity chromatography adsorbent column. The column was first washed with 5 column volumes (CV) of 0.5N NaOH, followed by 5 CV of autoclaved E-pure water. The column was then equilibrated with 10 CV of 50 mM sodium citrate, pH 5.5 (Buffer A). The protein fraction was then loaded onto the column, and subsequently washed with 10 CV of Buffer A. The purified protein was then eluted off the column with 5 CV of 50 mM ammonium acetate, 10 mM sodium octanoate, pH 7.0.

SDS-PAGE

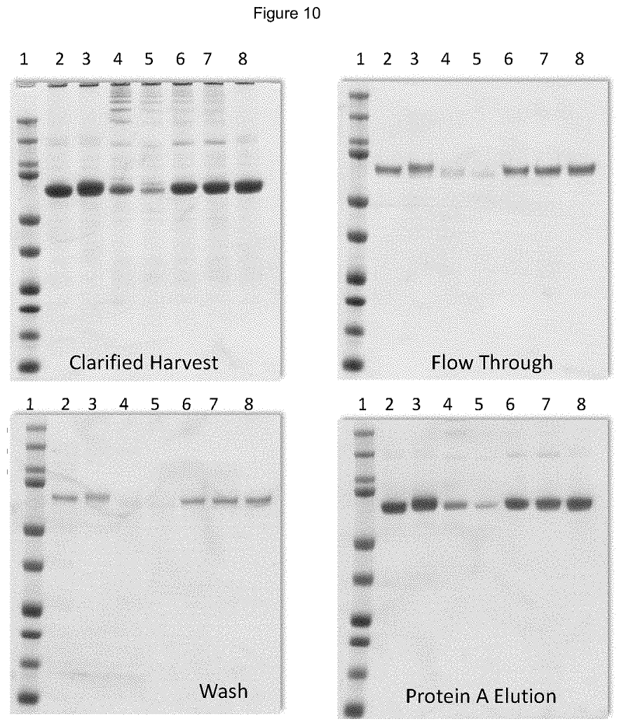

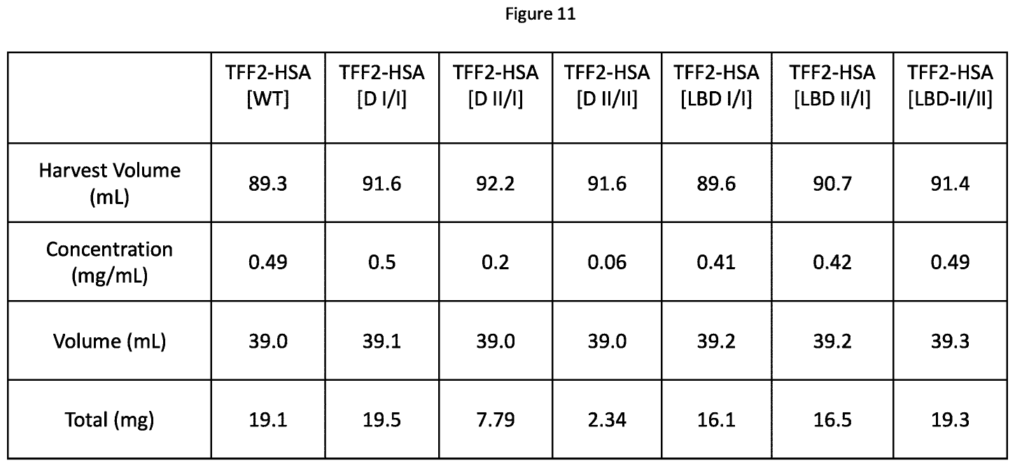

The samples were run on a NuPAGE Gel 4-12% Bis-Tris 1.0 mm, 12-well (Invitrogen®, cat # NPO302BOX). The samples (2 μg) were loaded in NuPAGE LDS sample buffer (4X), and run in MES buffer (Invitrogen®, cat # NP002-02) at 200V for 30 minutes. Precision Plus MW standards were used as molecular weight standards (Bio-Rad®, cat #161-0374). The gel was stained with Simply Blue Stain (Invitrogen®, cat # LC6060). The clarified harvest, the flow-through, the wash and the protein A elution samples were run in the gel. See FIG. 10. The yield obtained for each of the purified TFF2-HAS variants is shown in FIG. 11.

All patents, patent applications and publications, and non-patent publications cited herein are hereby incorporated by reference in their entirety.

A number of embodiments of the disclosure have been described. Nevertheless, it will be understood that various modifications may be made without departing from the spirit and scope of the present disclosure. Accordingly, other embodiments are within the scope of the following claims.

1. A composition comprising a modified TFF2 polypeptide, wherein the TFF2 polypeptide is modified by one or more of PEGylation, polysialylation, poly (D,L-lactic-co-glycolic acid) (PLGA)-conjugation and/or a fusion protein comprising a C-terminal peptide (CTP) of human chorionic gonadotropin β subunit, PASylation, XTENylation, ELPylation, or HAPylation.

2. The composition of claim 1, wherein the TFF2 polypeptide has a polypeptide sequence:
(a) that has at least 90% amino acid sequence identity to the amino acid sequence of SEQ ID NO: 1, SEQ ID NO: 3, or SEQ ID NO: 6
(b) that has at least 95% amino acid sequence identity to the amino acid sequence of SEQ ID NO: 1, SEQ ID NO: 3, or SEQ ID NO: 6: or
(c) of SEQ ID NO:1, SEQ ID NO: 3, or SEQ ID NO: 6.

3. (canceled)

4. (canceled)

5. The composition of claim 1, wherein the modified TFF2 polypeptide is PEGylated with a low molecular weight linear PEG or a high molecular weight branched PEG.

6.-7. (canceled)

8. The composition of claim 1, wherein the modified TFF2 polypeptide is a homogeneous population selected from the group consisting of a PEGylated TFF2 polypeptide, a polysialylated TFF2 polypeptide, a PLGA-conjugated TFF2 polypeptide, and a TFF2 polypeptide fusion protein comprising a CTP of human chorionic gonadotropin β subunit, PASylation, XTENylation, ELPylation, and HAPylation or combinations thereof.

9. The composition of claim 1, wherein the modified TFF2 polypeptide has increased half-life in blood as compared to unmodified human TFF2 polypeptide.

10. The composition of claim 1, wherein the modified TFF2 polypeptide is PEGylated at a specific site or sites.

11. The composition of claim 10, wherein the modified TFF2 peptide is PEGylated at its N-terminus or C-terminus.

12.-15. (canceled)

16. A method of treating cancer in a subject in need thereof comprising administering to the subject an effective amount of the composition of claim 1.

17. The method of claim 16, wherein the cancer is a cancer of the digestive system.

18. The method of claim 17, wherein the digestive cancer is selected from one or more of mouth cancer, pharynx cancer, oropharynx, esophageal cancer, gastric cancer, stomach cancer, small intestine cancer, large intestine cancer, colon cancer, rectal cancer, anal cancer, liver cancer, pancreatic cancer, and gall bladder cancer.

19.-26. (canceled)

27. The method of claim 16, further comprising treating the cancer with a blocking antibody to PD-1, PD-L1, or CTLA-4.

28. A method of treating cancer in a subject in need thereof comprising administering to the subject an effective amount of the composition of claim 1, wherein the cancer is non-responsive to treatment with a blocking antibody to PD-1, PD-L1, or CTLA, wherein the cancer becomes susceptible to treatment with a blocking antibody to PD-1, PD-L1, or CTLA-4 after treatment with the composition; and wherein the subject is subsequently treated with a blocking antibody to PD-1, PD-L1, or CTLA-4 within about 1 to about 60 days after administering the composition.

29. A modified TFF2 polypeptide wherein the modified TFF2 polypeptide comprises a fusion protein.

30. The modified TFF2 polypeptide of claim 29, wherein the fusion protein is selected from one or more of the group consisting of a TFF2-albumin protein, TFF2-IgG1 fusion protein, and TFF2-poly-histidine tag.

31. The modified TFF2 polypeptide of claim 30, wherein the fusion protein is a poly-histidine tag.

32. The modified TFF2 polypeptide of claim 31, wherein the histidine tag contains an amino-acid cleavage site.

33. The modified TFF2 polypeptide of claim 32, wherein the amino acid cleavage site is selected from SEQ ID NO: 20, SEQ ID NO: 21, SEQ ID NO: 22 and SEQ ID NO:23.

34.-36. (canceled)

37. A method of treating cancer in a subject in need thereof comprising administering to the subject an effective amount of one or more of the modified TFF2 polypeptides of claim 29.

38. The method of claim 37, wherein the cancer is a cancer of the digestive system.

39. The method of claim 38, wherein the digestive cancer is selected from one or more of mouth cancer, pharynx cancer, oropharynx, esophageal cancer, stomach cancer, small intestine cancer, large intestine cancer, colon cancer, gastric cancer, rectal cancer, anal cancer, liver cancer, pancreatic cancer, and gall bladder cancer.

40.-47. (canceled)

48. The method of claim 37, further comprising treating the cancer with a blocking antibody to PD-1, PD-L1, or CTLA-4.

49. A method of treating cancer in a subject in need thereof comprising administering to the subject an effective amount of the modified TFF2 polypeptide of claim 29, wherein the cancer is non-responsive to treatment with a blocking antibody to PD-1, PD-L1, or CTLA-4; wherein the cancer becomes susceptible to treatment with a blocking antibody to PD-1, PD-L1, or CTLA-4 after treatment with the modified TFF2 polypeptide; and wherein the subject is subsequently treated with a blocking antibody to PD-1, PD-L1, or CTLA-4 within about 1 to about 60 days after treatment with the modified TFF2 polypeptide.

50. A method for treating Inflammatory Bowel Disease (IBD) in a subject in need thereof, wherein the subject is administered with an effective amount of one or more compositions of claim 1.

51. The method of claim 50, wherein the IBD is Crohn's disease or Ulcerative Colitis.

52. The method of claim 50, wherein the composition is administered orally, intravenously, or intramuscularly.

53. A modified TFF2 polypeptide comprising:
a) one or more domain I binding-domains, wherein the one of more domain I binding-domain comprises SEQ ID NO: 24, wherein the polypeptide comprising one or more domain I binding-domains contains no domain II binding domain,
(b) one or more domain II binding-domains, wherein the one or more domain II binding-domain comprises SEQ ID NO: 25, and wherein the polypeptide comprising one or more domain II binding-domains contains no domain I binding domain;
(c) two domain I binding-domains, as set forth in SEQ ID NO: 26;
(d) two domain II binding domains, as set forth in SEQ ID NO: 27:
(e) a domain I binding domain and a domain II binding domain that are interchanged with each other and comprises the sequence set forth in SEQ ID NO: 28:
(f) amino acid substitutions in the receptor-binding site residues and comprising the sequence SEQ ID NO:29:
(g) amino acid substitutions in the receptor-binding site residues and comprising the sequence SEQ ID NO: 30: or
(h) amino acid substitutions in the receptor-binding site residues and comprising the sequence SEQ ID NO: 31.

54.-60. (canceled)

61. The modified TFF2 polypeptide of claim 53, wherein the TFF2 binding domain is further modified by one or more of PEGylation, polysialylation, conjugation with poly(D,L-lactic-co-glycolic acid) (PLGA) and/or expressed as a fusion protein, comprising fusion polypeptides selected from the group consisting of a C-terminal peptide (CTP) of human chorionic gonadotropin β subunit, a PASylated fusion polypeptide, a XTENylated fusion polypeptide, a ELPylated fusion polypeptide, and a HAPylated fusion polypeptide.

62. The modified TFF2 polypeptide of claim 61, wherein the modified TFF2 binding domain is PEGylated with a low molecular weight linear PEG or a high molecular weight branched PEG.

63.-64. (canceled)

65. The modified TFF2 polypeptide of claim 61 wherein the modified TFF2 binding domain is PEGylated at its N-terminus or C-terminus.

66.-70. (canceled)

71. The modified TFF2 polypeptide of claim 53, wherein the modified TFF2 polypeptide has increased half-life in blood as compared to a human wild-type TFF2 polypeptide of SEQ ID NO: 6.

72. The modified TFF2 polypeptide of claim 53, wherein a C-terminal peptide (CTP) of human chorionic gonadotropin is used to improve the pharmacokinetic (PK) and pharmacodynamic (PD) properties of the modified TFF2 polypeptide.

73. The modified TFF2 polypeptide of claim 53, wherein the modified TFF2 polypeptide is glycosylated.

74. A method of treating cancer in a subject in need thereof comprising administering to the subject an effective amount of one or more of the modified TFF2 polypeptides of claim 53.

75.-85. (canceled)

86. A method of treating cancer in a subject in need thereof comprising administering to the subject one or more modified TFF2 polypeptides of claim 53, wherein the cancer is non-responsive to treatment with a blocking antibody to PD-1, PD-L1, or CTLA-4, wherein the cancer becomes susceptible to treatment with a blocking antibody to PD-1, PD-L1, or CTLA-4 after treatment with the modified TFF2 polypeptide, and wherein the subject is subsequently treated with a blocking antibody to PD-1, PD-L1, or CTLA-4 within about 1 to about 60 days after treatment with the modified TFF2 polypeptide.

87. A method for treating Inflammatory Bowel Disease (IBD) in a subject in need thereof wherein the subject is administered with an effective amount of one or more modified TFF2 polypeptides of claim 53.

88.-89. (canceled)

90. A method for treating COVID-19 in a subject in need thereof, the method comprising administering to the subject one or more compositions of claim 1, one or more of the modified TFF2 polypeptide of claim 29, or one or more of the modified TFF2 polypeptide of claim 53.

91. The method of claim 90, wherein the composition or modified TFF2 polypeptide is administered orally, intravenously, or intramuscularly.

92. The method of claim 90, further comprising administering an agent that inhibits or reduces SARS-CoV-2 replication.

93. The method according to claim 90, further comprising administering an antiviral agent selected from the group consisting of ribavirin, interferon (alfacon-1), chloroquine, hydroxychloroquine, EIDD-2801, EIDD-1931, GS-5734, GS-441524, ivermectin, favipiravir, indomethacin, chlorpromazine, penciclovir, nafomostat, camostat, nitazoxanide, remdesivir, famotidine and dexamethasone.


SEQUENCE LISTING (39 sequences)

SEQ ID NO: 1 - protein, 129 aa, Homo sapiens
MGRRDAQLLAALLVLGLCALAGSEKPSPCQCSRLSPHNRTNCGFPGITSDQCFDNGCCFDSSVTGVPWCFHPLPKQESDQCVMEVSDRRNCGYPGISPEECASRKCCFSNFIFEVPWCFFPKSVEDCHY

SEQ ID NO: 2 - DNA, 717 bp, Homo sapiens
cacggtggaagggctggggccacggggcagagaagaaaggttatctctgcttgttggacaaacagaggggagattataaaacatacccggcagtggacaccatgcattctgcaagccaccctggggtgcagctgagctagacatgggacggcgagacgcccagctcctggcagcgctcctcgtcctggggctatgtgccctggcggggagtgagaaaccctccccctgccagtgctccaggctgagcccccataacaggacgaactgcggcttccctggaatcaccagtgaccagtgttttgacaatggatgctgtttcgactccagtgtcactggggtcccctggtgtttccaccccctcccaaagcaagagtcggatcagtgcgtcatggaggtctcagaccgaagaaactgtggctacccgggcatcagccccgaggaatgcgcctctcggaagtgctgcttctccaacttcatctttgaagtgccctggtgcttcttcccgaagtctgtggaagactgccattactaagagaggctggttccagaggatgcatctggctcaccgggtgttccgaaaccaaagaagaaacttcgccttatcagcttcatacttcatgaaatcctgggttttcttaaccatcttttcctcattttcaatggtttaacatataatttctttaaataaaacccttaaaatctgctaaaaaaaaaaaa

SEQ ID NO: 3 - protein, 129 aa, Mus musculus
MRPRGAPLLAVVLVLGLHALVEGEKPSPCRCSRLTPHNRKNCGFPGITSEQCFDLGCCFDSSVAGVPWCFHPLPNQESEQCVMEVSARKNCGYPGISPEDCASRNCCFSNLIFEVPWCFFPQSVEDCHY

SEQ ID NO: 4 - DNA, 571 bp, Mus musculus
attctgcaggctgcccaggtccagtggagcagacatgcgacctcgaggtgcccccctgctggcagtggtcctggttttgggactgcatgctctggtagagggcgagaaaccttccccctgtcggtgctccaggctgacaccccacaacagaaagaactgtggcttcccgggcatcaccagtgagcagtgctttgatcttggatgctgctttgactctagcgtcgctggggtcccttggtgtttccacccacttccaaaccaagaatcggagcagtgtgtcatggaagtgtcagctcgcaagaattgtgggtacccgggcatcagtcccgaggactgtgccagtcgaaactgctgcttttccaacctgatctttgaagtgccctggtgtttcttcccacagtctgtggaagattgtcactactgagagttgctactgccgagccacccgttccctgggagctgcaagccagaagaaagtttcaaccagacttcatcaatctctggggtttctaaaaccatcttgacccttagcagtggctagacacagcattttccaagtaaagaaaagttg

SEQ ID NO: 5 - skipped

SEQ ID NO: 6 - protein, 106 aa, Homo sapiens
EKPSPCQCSRLSPHNRTNCGFPGITSDQCFDNGCCFDSSVTGVPWCFHPLPKQESDQCVMEVSDRRNCGYPGISPEECASRKCCFSNFIFEVPWCFFPKSVEDCHY

SEQ ID NO: 7 - skipped

SEQ ID NO: 8 - protein, 6 aa, Artificial Sequence
  source: /note=“Description of Artificial Sequence: Synthetic 6xHis tag”
HHHHHH

SEQ ID NO: 9-10 - skipped (2 entries)

SEQ ID NO: 11 - protein, 30 aa, Unknown
  source: /note=“Description of Unknown: CTP peptide sequence”
FQSSSSKAPPPSLPSPSRLPGPSDTPILPQ

SEQ ID NO: 12 - protein, 5 aa, Artificial Sequence
  source: /note=“Description of Artificial Sequence: Synthetic peptide”
  MOD_RES (4)..(4): Any amino acid except Proline
VPGXG

SEQ ID NO: 13 - protein, 5 aa, Artificial Sequence
  source: /note=“Description of Artificial Sequence: Synthetic peptide”
  source: /note=“See specification as filed for detailed description of substitutions and preferred embodiments”
GGGGS

SEQ ID NO: 14 - protein, 22 aa, Homo sapiens
MDMRVLAQLLGLLLLCFPGARA

SEQ ID NO: 15 - protein, 18 aa, Homo sapiens
MKVLWAALLVTFLAGCQA

SEQ ID NO: 16 - protein, 26 aa, Bos sp.
MMAAGPRTSLLLAFALLCLPWTQVVG

SEQ ID NO: 17 - protein, 23 aa, Drosophila sp.
MKLIAVTIIACILLIGFSDLALG

SEQ ID NO: 18 - protein, 24 aa, Homo sapiens
MKWVTFISLLFLFSSAYSRGVFRR

SEQ ID NO: 19 - protein, 21 aa, Homo sapiens
MSMLVVFLLLWGVTWGPVTEA

SEQ ID NO: 20 - protein, 6 aa, Unknown
  source: /note=“Description of Unknown: cleavage site peptide”
LVPRGS

SEQ ID NO: 21 - protein, 4 aa, Unknown
  source: /note=“Description of Unknown: cleavage site peptide”
DDDK

SEQ ID NO: 22 - protein, 7 aa, Unknown
  source: /note=“Description of Unknown: cleavage site peptide”
ENLYFQG

SEQ ID NO: 23 - protein, 7 aa, Unknown
  source: /note=“Description of Unknown: cleavage site peptide”
ENLYFQS

SEQ ID NO: 24 - protein, 39 aa, Homo sapiens
CSRLSPHNRTNCGFPGITSDQCFDNGCCFDSSVTGVPWC

SEQ ID NO: 25 - protein, 38 aa, Homo sapiens
CVMEVSDRRNCGYPGISPEECASRKCCFSNFIFEVPWC

SEQ ID NO: 26 - protein, 107 aa, Artificial Sequence
  source: /note=“Description of Artificial Sequence: Synthetic polypeptide”
EKPSPCQCSRLSPHNRTNCGFPGITSDQCFDNGCCFDSSVTGVPWCFHPLPKQESDQCSRLSPHNRTNCGFPGITSDQCFDNGCCFDSSVTGVPWCFFPKSVEDCHY

SEQ ID NO: 27 - protein, 105 aa, Artificial Sequence
  source: /note=“Description of Artificial Sequence: Synthetic polypeptide”
EKPSPCQCVMEVSDRRNCGYPGISPEECASRKCCFSNFIFEVPWCFHPLPKQESDQCVMEVSDRRNCGYPGISPEECASRKCCFSNFIFEVPWCFFPKSVEDCHY

SEQ ID NO: 28 - protein, 106 aa, Artificial Sequence
  source: /note=“Description of Artificial Sequence: Synthetic polypeptide”
EKPSPCQCVMEVSDRRNCGYPGISPEECASRKCCFSNFIFEVPWCFHPLPKQESDQCSRLSPHNRTNCGFPGITSDQCFDNGCCFDSSVTGVPWCFFPKSVEDCHY

SEQ ID NO: 29 - protein, 106 aa, Artificial Sequence
  source: /note=“Description of Artificial Sequence: Synthetic polypeptide”
EKPSPCQCSRLSPHNRTNCGYPGISSEECFDRGCCFDSSVTGVPWCFHPLPKQESDQCVMEVSDRRNCGFPGITPDQCASNKCCFSNFIFEVPWCFFPKSVEDCHY

SEQ ID NO: 30 - protein, 106 aa, Artificial Sequence
  source: /note=“Description of Artificial Sequence: Synthetic polypeptide”
EKPSPCQCSRLSPHNRTNCGFPGITSDQCFDNGCCFDSSVTGVPWCFHPLPKQESDQCVMEVSDRRNCGFPGITPDQCASNKCCFSNFIFEVPWCFFPKSVEDCHY

SEQ ID NO: 31 - protein, 106 aa, Artificial Sequence
  source: /note=“Description of Artificial Sequence: Synthetic polypeptide”
EKPSPCQCSRLSPHNRTNCGYPGISSEECFDRGCCFDSSVTGVPWCFHPLPKQESDQCVMEVSDRRNCGYPGISPEECASRKCCFSNFIFEVPWCFFPKSVEDCHY

SEQ ID NO: 32 - DNA, 507 bp, Artificial Sequence
  source: /note=“Description of Artificial Sequence: Synthetic polynucleotide”
atgggcagaagagacgcacagctattagctgctctgctggtgttaggactgtgtgctttggctggatctgagaagccttctccttgccagtgttctagactgagcccccacaataggaccaattgcggctttccaggcatcacctctgatcagtgcttcgataatggctgctgcttcgatagcagcgttacaggcgttccttggtgcttccatcctctgcctaaacaggaaagcgatcagtgcgtgatggaggtgtctgacagaaggaattgcggctatcctggcatctctcctgaagaatgtgccagcaggaagtgctgcttcagcaacttcatcttcgaggttccttggtgcttcttccccaagtctgtggaggactgccactacgagaacctgtactttcaaggaggaggaggaggaggatctcaccaccatcaccaccaccaccaccatcatggaggaggaggatctggaggatcttggtctcatcctcagtttgagaagtag

SEQ ID NO: 33 - protein, 168 aa, Artificial Sequence
  source: /note=“Description of Artificial Sequence: Synthetic polypeptide”
MGRRDAQLLAALLVLGLCALAGSEKPSPCQCSRLSPHNRTNCGFPGITSDQCFDNGCCFDSSVTGVPWCFHPLPKQESDQCVMEVSDRRNCGYPGISPEECASRKCCFSNFIFEVPWCFFPKSVEDCHYENLYFQGGGGGGSHHHHHHHHHHGGGGSGGSWSHPQFEK

SEQ ID NO: 34 - DNA, 1404 bp, Artificial Sequence
  source: /note=“Description of Artificial Sequence: Synthetic polynucleotide”
atgggcagaagagacgcacagctattagctgctctgctggtgttaggactgtgtgctttggctggatctgagaagccttctccttgccagtgttctagactgagcccccacaataggaccaattgcggctttccaggcatcacctctgatcagtgcttcgataatggctgctgcttcgatagcagcgttacaggcgttccttggtgcttccatcctctgcctaaacaggaaagcgatcagtgcgtgatggaggtgtctgacagaaggaattgcggctatcctggcatctctcctgaagaatgtgccagcaggaagtgctgcttcagcaacttcatcttcgaggttccttggtgcttcttccccaagtctgtggaggactgccactatggaggaggaggatctggaggatctgctagcacaaaaggacctagcgtttttcctctggccccatctagcaagagcacatctggcggaacagctgctttgggatgtctggtgaaggattactttcccgagcctgtgacagtgagctggaattctggagccctgacatctggagtgcacacctttcctgctgttctgcagtcttctggcctgtattctctgtctagcgtggtgacagtgcctagctcttctctgggaacacagacctacatctgcaacgtgaaccacaagcccagcaacaccaaggtggacaagaaagtggagcctaagagctgcgataagacccacacatgtcctccatgtcctgcccctgaactgttaggaggacctagcgttttcctgtttccacctaagcccaaagataccctgatgatcagcaggacccctgaggtgacctgtgtggtggttgatgtgagccatgaggatcctgaagtgaagttcaactggtacgtggatggcgtggaagtgcacaacgccaagaccaagcctagagaagagcagtacaatagcacctacagagtggtgagcgtgctgacagtgctgcaccaggattggctgaatggcaaggagtataagtgcaaggtgagcaataaggccctgccagcccctatcgagaagaccatctctaaggccaagggacaacctagagaaccacaggtttacacactgccccccagcagagatgagctgaccaaaaaccaggtgtctctgacatgtctggtgaagggcttttatcccagcgacatcgccgtggaatgggagtctaatggacagcccgagaataactacaagaccacacctccagtgctggatagcgatggcagcttcttcctgtacagcaagctgaccgtggataaaagcagatggcaacagggcaacgtgtttagctgcagcgtgatgcatgaagccctgcacaaccactatacccagaaaagcctgagcctgtctcctggcaagtaa

SEQ ID NO: 35 - protein, 467 aa, Artificial Sequence
  source: /note=“Description of Artificial Sequence: Synthetic polypeptide”
MGRRDAQLLAALLVLGLCALAGSEKPSPCQCSRLSPHNRTNCGFPGITSDQCFDNGCCFDSSVTGVPWCFHPLPKQESDQCVMEVSDRRNCGYPGISPEECASRKCCFSNFIFEVPWCFFPKSVEDCHYGGGGSGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - DNA, 2163 bp, Artificial Sequence
  source: /note=“Description of Artificial Sequence: Synthetic polynucleotide”
atgggcagaagagacgcacagctattagctgctctgctggtgttaggactgtgtgctttggctggatctgagaagccttctccttgccagtgttctagactgagcccccacaataggaccaattgcggctttccaggcatcacctctgatcagtgcttcgataatggctgctgcttcgatagcagcgttacaggcgttccttggtgcttccatcctctgcctaaacaggaaagcgatcagtgcgtgatggaggtgtctgacagaaggaattgcggctatcctggcatctctcctgaagaatgtgccagcaggaagtgctgcttcagcaacttcatcttcgaggttccttggtgcttcttccccaagtctgtggaggactgccactatggaggaggaggatctgatgcccataaatctgaagtggcccacaggttcaaggatctgggagaggagaacttcaaggccctggtgctgatcgcttttgctcaatacctgcagcagtgcccttttgaggatcacgtgaaactggtgaacgaggtgaccgagtttgccaagacatgtgtggccgatgagtctgccgagaattgcgataaaagcctgcacaccctgttcggagacaagctgtgtacagtggctaccctgagagagacatatggcgaaatggccgattgttgcgccaaacaggaacccgagagaaatgagtgcttcctgcagcacaaggacgacaaccctaatctgcctaggctggttagacctgaggtggatgtgatgtgtaccgccttccacgacaatgaggagacattcctgaagaagtacctgtacgagatcgcccggagacacccttacttctacgcccctgaactgctgtttttcgccaagagatacaaagccgcctttaccgagtgctgtcaggctgccgataaagctgcctgtttactgcccaagctggatgaactgagagatgagggaaaggcctctagcgccaagcagagactgaaatgtgctagcctgcagaagtttggcgaaagagcctttaaagcctgggctgtggccagactgagccagagatttcctaaagccgagtttgccgaagtgagcaaattagtgaccgacctgaccaaggtgcacaccgagtgttgtcatggcgatcttctggaatgcgccgatgatagagctgatctggccaagtacatctgcgagaaccaggatagcatcagcagcaagctgaaggagtgttgcgagaaacctctgctggagaaaagccactgtatcgccgaagtggagaacgacgagatgcctgctgatctgccttctttagccgccgattttgtggagagcaaggatgtgtgcaagaactacgccgaggccaaagacgtgtttttgggcatgttcctgtacgagtacgccagaagacaccctgattatagcgtggtgctgctgctgagactggccaagacatacgagacaacactggagaagtgttgtgctgctgctgatcctcacgagtgttacgccaaggtgttcgacgagttcaaacctctggtggaagaacctcagaacctgatcaagcagaactgcgagctgttcgagcagctgggcgagtacaagttccagaatgctctgctggtgagatacaccaagaaagtgcctcaggtgtctacccccaccctggttgaagtgagcagaaatctgggcaaagtgggctctaaatgttgcaagcaccctgaggccaagaggatgccttgtgccgaggattatctgtctgtggtgctgaatcaactgtgtgtgctgcacgagaagacccctgtgagcgacagagtgacaaagtgttgtaccgagtctctggtgaacagaagaccctgcttttctgccctggaggtggatgagacctatgtgcctaaggagttcaatgccgagacctttaccttccatgccgacatctgcaccctgagcgagaaagagaggcagatcaagaaacagacagccctggttgaactggtgaagcacaagcctaaggccaccaaagagcagctgaaagccgttatggacgattttgccgcctttgtggagaagtgctgtaaggccgacgataaggagacctgtttcgccgaagagggaaaaaagctggttgctgcctctcaagctgctctgggcctgtaataa

SEQ ID NO: 37 - protein, 719 aa, Artificial Sequence
  source: /note=“Description of Artificial Sequence: Synthetic polypeptide”
MGRRDAQLLAALLVLGLCALAGSEKPSPCQCSRLSPHNRTNCGFPGITSDQCFDNGCCFDSSVTGVPWCFHPLPKQESDQCVMEVSDRRNCGYPGISPEECASRKCCFSNFIFEVPWCFFPKSVEDCHYGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 38 - DNA, 645 bp, Artificial Sequence
  source: /note=“Description of Artificial Sequence: Synthetic polynucleotide”
atgggcagaagagacgcacagctattagctgctctgctggtgttaggactgtgtgctttggctggatctgagaagccttctccttgccagtgttctagactgagcccccacaataggaccaattgcggctttccaggcatcacctctgatcagtgcttcgataatggctgctgcttcgatagcagcgttacaggcgttccttggtgcttccatcctctgcctaaacaggaaagcgatcagtgcgtgatggaggtgtctgacagaaggaattgcggctatcctggcatctctcctgaagaatgtgccagcaggaagtgctgcttcagcaacttcatcttcgaggttccttggtgcttcttccccaagtctgtggaggactgccactacagcagctcttctaaagctcctcctccttctctgccttctccttctagacttcctggccctagcgatacccctattctgcctcaaagcagcagctctaaagctcctcctccttctttacctagccccagcagacttcctggaccttctgatacccctatcctgcctcaaacaggcatggactataaggacgacgacgacaaggactacaaggacgacgacgacaaggactacaaggatgacgacgacaaagccagctaa

SEQ ID NO: 39 - protein, 214 aa, Artificial Sequence
  source: /note=“Description of Artificial Sequence: Synthetic polypeptide”
MGRRDAQLLAALLVLGLCALAGSEKPSPCQCSRLSPHNRTNCGFPGITSDQCFDNGCCFDSSVTGVPWCFHPLPKQESDQCVMEVSDRRNCGYPGISPEECASRKCCFSNFIFEVPWCFFPKSVEDCHYSSSSKAPPPSLPSPSRLPGPSDTPILPQSSSSKAPPPSLPSPSRLPGPSDTPILPQTGMDYKDDDDKDYKDDDDKDYKDDDDKAS